(19) United States
(12) Patent Application Publication
(10) Pub. No.: US 2025/0359727 A1
(43) Pub. Date: Nov. 27, 2025

(54) IMAGING APPARATUS AND ENDOSCOPIC SYSTEM

(71) Applicant: JVCKENWOOD Corporation, Yokohama-shi (JP)

(72) Inventors: Toshihide KOBAYASHI, Yokohama-shi (JP); Shogo MASUDA, Yokohama-shi (JP); Kazunori SHIODA, Yokohama-shi (JP)

(21) Appl. No.: 19/290,407

(22) Filed: Aug. 5, 2025

Related U.S. Application Data
(63) Continuation of application No. PCT/JP2024/003998, filed on Feb. 7, 2024.

(30) Foreign Application Priority Data
Feb. 16, 2023 (JP) 2023-022655
Feb. 16, 2023 (JP) 2023-022656

(51) Int. Cl. A61B 1/00; A61B 1/05; G06T 7/70; G06T 7/90; H04N 23/45; H04N 23/50; H04N 23/81; H04N 23/84

(52) U.S. Cl. CPC A61B 1/00009; A61B 1/051; G06T 7/70; G06T 7/90; H04N 23/45; H04N 23/555; H04N 23/81; H04N 23/843; G06T 2207/10024; G06T 2207/10028

(57) ABSTRACT

An imaging apparatus includes: a polarization image sensor for detecting four polarization components; an interpolation processing unit that calculates, pixel by pixel, an RGB value of each of the four polarization components from the polarization image sensor; a rank processing unit that ranks the four polarization components; a reference generation unit that calculates a reference brightness value; and a synthesis processing unit that a) outputs a reference RGB value when the reference brightness value matches a predetermined threshold value, b) outputs a composite RGB value obtained by synthesizing a high-rank RGB value and the reference RGB value when the reference brightness value is smaller than the threshold value, and c) outputs a composite RGB value obtained by synthesizing a low-rank RGB value and the reference RGB value when the reference brightness value is greater than the threshold value.

### CROSS REFERENCE TO RELATED APPLICATION

This application is a continuation of application No. PCT/JP2024/003998, filed on Feb. 7, 2024, and claims the benefit of priority from the prior Japanese Patent Application No. 2023-022655, filed on Feb. 16, 2023 and the prior Japanese Patent Application No. 2023-022656, filed on Feb. 16, 2023, the entire contents of which are incorporated herein by reference.

### BACKGROUND

### 1. Technical Field

The present disclosure relates to an imaging apparatus and an endoscopic system.

### 2. Description of the Related Art

An imaging apparatus for observing the polarization state of a light from a subject is known. For example, a technology is proposed in which a plurality of color images with different polarization states are acquired in a time divided manner, a composite image is generated by using polarization information on the subject based on the plurality of color images, and the color is corrected based on a reference image selected from the plurality of color images. A technology to suppress overexposure or underexposure by using, as the reference image, an HDR (High Dynamic Range) image produced by synthesizing a plurality of color images at different exposure conditions has been proposed (see, for example, Patent Literature 1).
[Patent Literature 1] JP 2017-191986 A

In the above-described technology, color images with different polarization states are acquired by using a variable phase difference plate so that coloring caused by the variable phase difference plate is produced.

### SUMMARY

An imaging apparatus according to an embodiment of the present disclosure includes: a polarization image sensor in which pixel groups, each including 2×2 pixels for detecting four polarization components that vary depending on a pixel, are in a two-dimensional arrangement, and in which RGB color filters are in a Bayer arrangement, each RGB filter being arranged in each pixel group, an interpolation processing unit that decomposes a pixel value output from the polarization image sensor into each polarization component, generates four Bayer images corresponding to the four polarization components, and calculates, pixel by pixel, an RGB value of each of the four polarization components by debayering and upconverting each of the four Bayer images; a brightness calculation unit that calculates, pixel by pixel, a brightness value of each of the four polarization components from the RGB value of each of the four polarization components; a rank processing unit that ranks, pixel by pixel, the four polarization components in an order of magnitude of the brightness values of the four polarization components; a reference generation unit that calculates, pixel by pixel, a reference brightness value obtained by synthesizing brightness values of a plurality of polarization components that at least include a second-place polarization component and a third-place polarization component; and a synthesis processing unit that a) outputs a reference RGB value obtained by synthesizing the RGB values of the plurality of polarization components when the reference brightness value matches a predetermined threshold value, b) outputs a composite RGB value obtained by synthesizing a high-rank RGB value, derived from synthesizing the RGB values of a first-place polarization component and the second-place polarization component, and the reference RGB value when the reference brightness value is smaller than the threshold value, and c) outputs a composite RGB value obtained by synthesizing a low-rank RGB value, derived from synthesizing the RGB values of the third-place polarization component and a fourth-place polarization component, and the reference RGB value when the reference brightness value is greater than the threshold value.

Another embodiment of the present disclosure relates to an endoscopic system including an imaging apparatus. The endoscopic system includes: an endoscope including an inserted portion having a tip portion directed toward a subject, the polarization image sensor being provided inside the tip portion, and a transmission cable for transmitting an output signal of the polarization image sensor being provided inside the inserted portion; and an image processing apparatus including the interpolation processing unit, the brightness calculation unit, the rank processing unit, the reference generation unit, and the synthesis processing unit, the image processing apparatus being configured to acquire the output signal via the transmission cable.

Optional combinations of the aforementioned constituting elements, and mutual substitution of constituting elements and implementations of the present disclosure between methods, apparatuses, systems, etc. may also be practiced as additional modes of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 schematically shows a configuration of an imaging apparatus according to the first embodiment;

FIG. 2 is a plan view schematically showing a configuration of the light detection layer of the polarization image sensor;

FIG. 3 is a plan view schematically showing a configuration of the polarizer layer of the polarization image sensor;

FIG. 4 is a plan view schematically showing a configuration of the color filter layer of the polarization image sensor;

FIG. 5 is a plan view schematically showing a configuration of the microlens layer of the polarization image sensor;

FIG. 6 schematically shows the flow of the image process performed by the interpolation processing unit;

FIG. 7 schematically shows the flow of the image process performed by the synthesis processing unit;

FIG. 8 schematically shows a configuration of an imaging apparatus according to the second embodiment;

FIG. 9 is a plan view schematically showing a configuration of the color filter layer of the non-polarization image sensor;

FIG. 10 is a graph showing an example of the frequency characteristics of the low-pass filter used in the high-frequency extraction unit;

FIG. 11 schematically shows the flow of the image process performed by the second processing unit;

FIG. 12 schematically shows a configuration of an imaging apparatus according to the third embodiment;

FIG. 13 schematically shows the flow of the image process performed by the second processing unit;

FIG. 14 schematically shows a configuration of an imaging apparatus according to the fourth embodiment;

FIG. 15 schematically shows a configuration of an imaging apparatus according to the fifth embodiment;

FIG. 16 schematically shows an imaging unit according to the first exemplary configuration of the fifth embodiment;

FIG. 17 schematically shows an imaging unit according to the second exemplary configuration of the fifth embodiment;

FIG. 18 schematically shows an imaging unit according to the third exemplary configuration of the fifth embodiment;

FIG. 19 schematically shows a configuration of an endoscopic system according to the sixth embodiment; and

FIG. 20 schematically shows a configuration of an endoscopic system according to the seventh embodiment.

### DETAILED DESCRIPTION

The invention will now be described by reference to the preferred embodiments. This does not intend to limit the scope of the present invention, but to exemplify the invention.

A description will be given below of embodiments of the present disclosure with reference to the drawings. Specific numerical values shown in the embodiments are by way of example only to facilitate the understanding of the invention and should not be construed as limiting the disclosure unless specifically indicated as such. Those elements in the drawings not directly relevant to the present disclosure are omitted from the illustration.

### First Embodiment

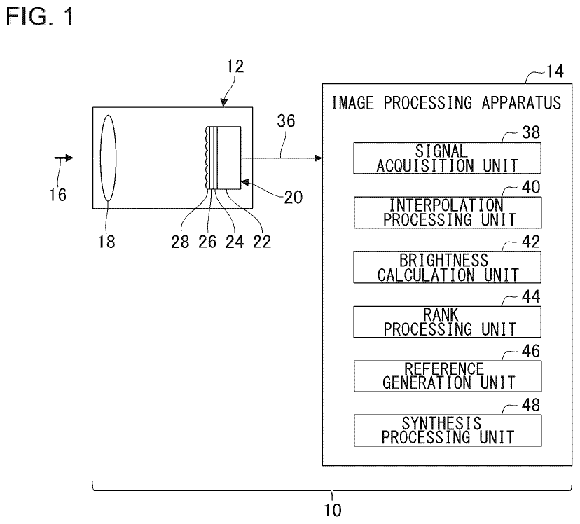

FIG. 1 schematically shows a configuration of an imaging apparatus 10 according to the first embodiment. The imaging apparatus 10 includes an imaging unit 12 and an image processing apparatus 14.

The imaging unit 12 includes an imaging lens 18 and a polarization image sensor 20.

The imaging lens 18 is provided in front of the polarization image sensor 20. The imaging lens 18 is arranged to form an image of an incident light 16 incident on the imaging unit 12 on the light receiving surface of the polarization image sensor 20. The imaging lens 18 may include one or more desired number of optical lenses.

The polarization image sensor 20 includes a plurality of pixels for imaging the incident light 16. The polarization image sensor 20 includes a light detection layer 22, a polarizer layer 24, a color filter layer 26, and a microlens layer 28. The light detection layer 22, the polarizer layer 24, the color filter layer 26, and the microlens layer 28 are arranged in alignment in the direction of incidence of the incident light 16. In the example of FIG. 1, the microlens layer 28, the color filter layer 26, the polarizer layer 24, and the light detection layer 22 are arranged in the stated order as seen in the direction of incidence of the incident light 16. The stacking order of the polarizer layer 24 and the color filter layer 26 does not matter. For example, the microlens layer 28, the polarizer layer 24, the color filter layer 26, and the light detection layer 22 may be stacked in the stated order.

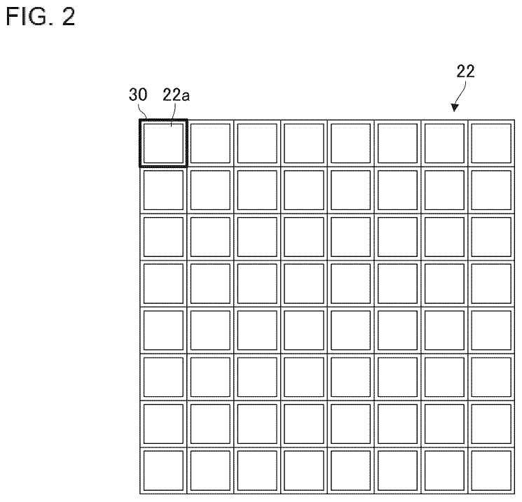

FIG. 2 is a plan view schematically showing a configuration of the light detection layer 22 of the polarization image sensor 20. The light detection layer 22 is, for example, configured in the same way as a two-dimensional image sensor such as a CCD (Charge Coupled Devices) sensor and a CMOS (Complementary Metal Oxide Semiconductor) sensor. The light detection layer 22 includes a photodiode 22a for detecting the incident light 16 and converting it into an electrical signal. The light detection layer 22 includes a plurality of photodiodes 22a in a two-dimensional arrangement. The light detection layer 22 includes, for example, one photodiode 22a for each pixel 30 of the polarization image sensor 20.

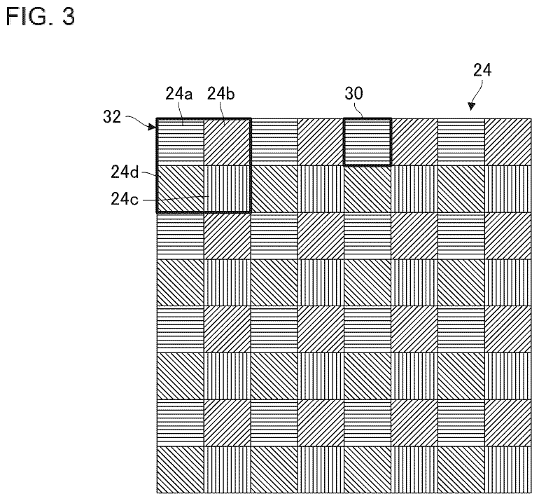

FIG. 3 is a plan view schematically showing a configuration of the polarizer layer 24 of the polarization image sensor 20. The polarizer layer 24 includes a first polarizer 24a, a second polarizer 24b, a third polarizer 24c, and a fourth polarizer 24d for detecting polarization components that vary depending on the pixel 30. In other words, one of the four polarizers 24a-24d is provided in one pixel 30. The first polarizer 24a selectively transmits the first polarizing component that is linearly polarized in the first direction (e.g., horizontal or 0-degree direction). The second polarizer 24b selectively transmits the second polarization component that is linearly polarized in the second direction (e.g., diagonally rightward or 45-degree direction). The third polarizer 24c selectively transmits the third polarization component that is linearly polarized in the third direction (e.g., vertical or 90-degree direction). The fourth polarizer 24d selectively transmits the fourth polarization component that is linearly polarized in the fourth direction (e.g., diagonally leftward or 135-degree direction). The polarizers 24a-24d are, for example, wire grid polarizers.

The polarizer layer 24 has a structure in which pixel groups 32 each including four pixels of 2×2 in the height and width are in a two-dimensional arrangement as repeating units. One pixel group 32 includes a first pixel provided with the first polarizer 24a, a second pixel provided with the second polarizer 24b, a third pixel provided with the third polarizer 24c, and a fourth pixel provided with the fourth polarizer 24d. The first polarizer 24a and the third polarizer 24c are provided in diagonal pixels in one pixel group 32. The second polarizer 24b and the fourth polarizer 24d are provided in diagonal pixels in one pixel group 32. Each of the four polarizers 24a-24d is placed in every other pixel in the height and width in a two-dimensional arrangement.

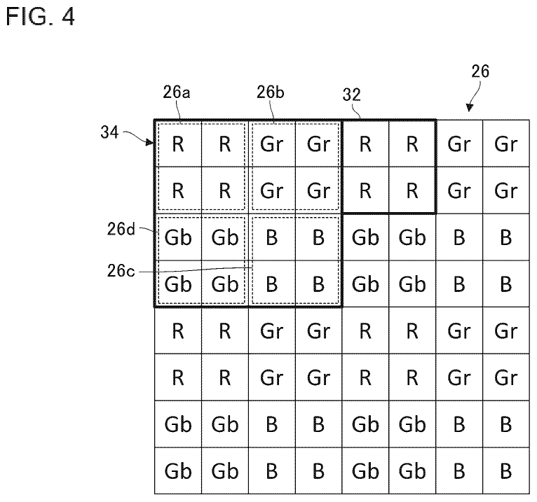

FIG. 4 is a plan view schematically showing a configuration of the color filter layer 26 of the polarization image sensor 20. The color filter layer 26 includes a red (R) filter 26a, a green (Gr) filter 26b, a blue (B) filter 26c, or a green (Gb) filter 26d in each pixel group 32, the RGB filters being in the Bayer arrangement. In other words, one of the four color filters 26a-26d is provided in one pixel group 32. Each of the four color filters 26a-26d is arranged to occupy 4 pixels of 2×2 in the height and width. The color filter layer 26 has a structure in which pixel sets 34 each including four vertically and horizontally adjacent pixel groups 32 are in a two-dimensional arrangement as repeating units. The pixel set 34 includes 16 pixels of 4×4 in the height and width.

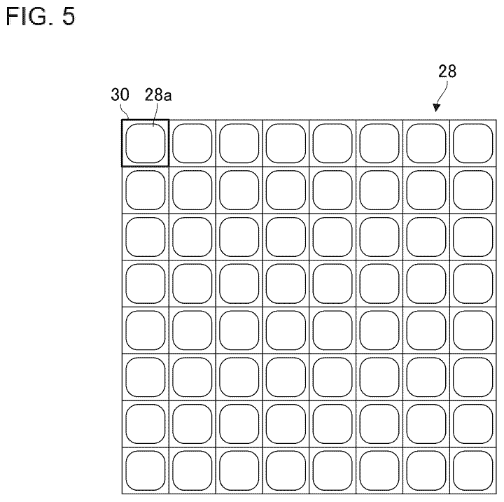

FIG. 5 is a plan view schematically showing a configuration of the microlens layer 28 of the polarization image sensor 20. The microlens layer 28 includes a plurality of microlenses 28a in a two-dimensional arrangement. The microlens layer 28 includes, for example, one microlens 28a for each pixel 30 of the polarization image sensor 20.

Referring back to FIG. 1, the image processing apparatus 14 will be described. The image processing apparatus 14 generates an image by using an output signal of the polarization image sensor 20. The image processing apparatus 14 includes a signal acquisition unit 38, an interpolation processing unit 40, a brightness calculation unit 42, a rank processing unit 44, a reference generation unit 46, and a synthesis processing unit 48.

The image processing apparatus 14 may, for example, be configured by an electronic circuit such as a DSP (Digital Signal Processor) or ISP (Image Signal Processor) for executing hardware-based signal processing or image processing. Each functional block constituting the image processing apparatus 14 can be configured by one or more electronic circuits. The image processing apparatus 14 may be implemented by a combination of hardware and software. The hardware of the image processing apparatus 14 may be implemented by devices and mechanical apparatus exemplified by a processor such as a CPU (Central Processing Unit) and a GPU (Graphics Processing Unit) and by a memory such as a ROM (Read Only Memory) and a RAM (Random Access Memory). The software of the image processing apparatus 14 may be implemented by a computer program, etc. In this case, the image processing apparatus 14 is depicted as a functional block implemented by cooperation of hardware and software. It will be understood by those skilled in the art that the functional blocks of the image processing apparatus 14 can be implemented in a variety of manners by a combination of hardware and software.

The signal acquisition unit 38 acquires an image signal 36 output from the polarization image sensor 20. The image signal 36 corresponds to raw data output from the polarization image sensor 20. For example, the image signal 36 is serial data for the pixel value of each pixel 30 read in the order of address of each pixel 30 of the polarization image sensor 20. The number of bits of the pixel value of the image signal 36 does not particularly matter. For example, the pixel value includes 12 bits.

The interpolation processing unit 40 generates a color image of each of the four polarization components from the image signal 36 acquired by the signal acquisition unit 38. The interpolation processing unit 40 decomposes the pixel value included in the image signal 36 into each polarization component and generates four Bayer images corresponding to the four polarization components. The interpolation processing unit 40 generates four up-converted images in which the RGB value of each of the four polarization components are set for each pixel by debayering (Bayer transform) and upconverting (horizontal and vertical interpolation) each of the four Bayer images.

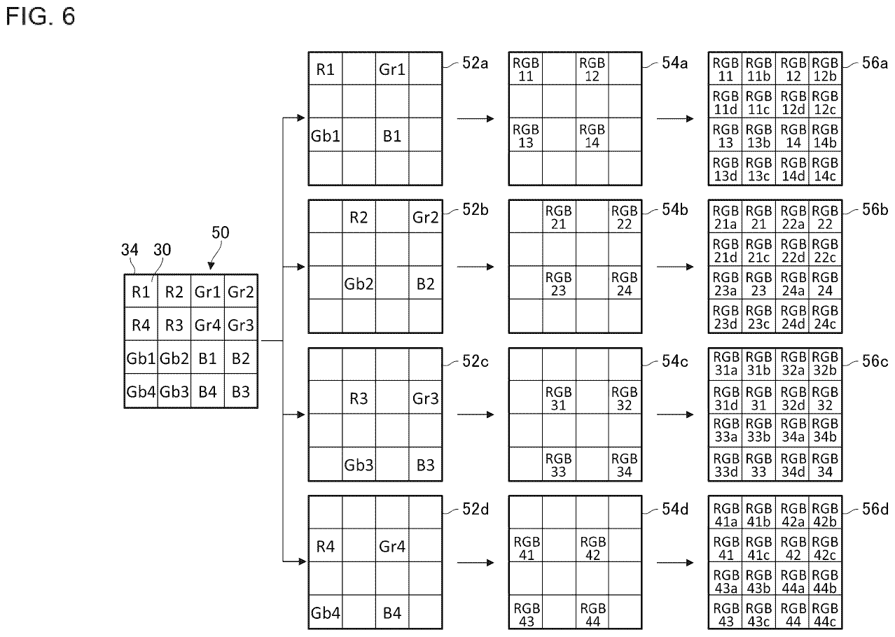

FIG. 6 schematically shows the flow of the image process performed by the interpolation processing unit 40. FIG. 6 shows a clip of only 16 pixels of 4×4 in the height and width constituting one pixel set 34 in order to simplify the explanation. The input image 50 corresponds to RAW data based on the image signal 36, and only one pixel value is stored in each pixel 30. One pixel set 34 includes 16 pixels corresponding to a combination of four colors (R, Gr, Gb, B) and four polarization components (1, 2, 3, 4). For example, the pixel value R1 indicates the pixel value of the pixel of the first polarizing component (1) of red (R) passing through the R filter 26a and the first polarizer 24a.

The interpolation processing unit 40 decomposes the input image 50 into four Bayer images 52a, 52b, 52c, and 52d for the respective polarization components. The first Bayer image 52a is composed of pixel values R1, Gr1, Gb1, and B1 of the first polarization component of four colors (R, Gr, Gb, B). The positions (phases) of the respective pixel values R1, Gr1, Gb1, and B1 are common to the input image 50. The second Bayer image 52b is composed of pixel values R2, Gr2, Gb2, and B2 of the second polarization component of four colors (R, Gr, Gb, B). The positions (phases) of the respective pixel values R2, Gr2, Gb2, and B2 are common to the input image 50. The third Bayer image 52c is composed of pixel values R3, Gr3, Gb3, and B3 of the third polarization component of four colors (R, Gr, Gb, B). The positions (phases) of the respective pixel values R3, Gr3, Gb3, and B3 are common to the input image 50. The fourth Bayer image 52d is composed of pixel values R4, Gr4, Gb4, and B4 of the fourth polarization component of four colors (R, Gr, Gb, B). The positions (phases) of the respective pixel values R4, Gr4, Gb4, and B4 are common to those of the input image 50.

The interpolation processing unit 40 debayers each of the four Bayer images 52a-52d to generate debayered images 54a, 54b, 54c, and 54d in which one RGB value is stored for each of 2×2 pixels (i.e., the pixel group 32). The first debayered image 54a is generated by debayering the first Bayer image 52a. Any known technology can be used in the debayering process. The first debayered image 54a is composed of RGB values RGB11, RGB12, RGB13, and RGB14 each of which corresponds to the position (phase) of the pixel in the pixel group 32 provided with the first polarizer 24a. The RGB value is represented in array data provided with pixel values (R, G, B) for R, G, and B.

“RGB” in the following expressions principally means, unless otherwise specified, calculation based on an array (R, G, B) including an R value, a G value and a B value, i.e., calculation made for each of the R value, the G value and B the value. In other words, the notation “RGB” is used for the purpose of simplifying the notation for the calculation of each of the R value, the G value, and the B value.

The second debayered image 54b is generated by debayering the second Bayer image 52b. The second debayered image 54b is composed of RGB values RGB21, RGB22, RGB23, and RGB24 each of which corresponds to the position (phase) of the pixel in the pixel group 32 provided with the second polarizer 24b. The third debayered image 54c is generated by debayering the third Bayer image 52c. The third debayered image 54c is composed of RGB values RGB31, RGB32, RGB33, and RGB34 each of which corresponds to the position (phase) of the pixel in the pixel group 32 provided with the third polarizer 24c. The fourth debayered image 54d is generated by debayering the fourth Bayer image 52d. The fourth debayered image 54d is composed of RGB values RGB41, RGB42, RGB43, and RGB44 each of which corresponds to the position (phase) of the pixel in the pixel group 32 provided with the fourth polarizer 24d.

The interpolation processing unit 40 generates four upconverted images 56a, 56b, 56c, and 56d by upconverting each of the four debayered images 54a-54d. The interpolation processing unit 40 generates four up-converted images 56a-56d by interpolating the pixel values of each of the four debayered images 54a-54d in the horizontal and vertical directions. Any known technology can be used in the upconversion process. In the example of FIG. 6, upconverting by a factor of 2 is carried out vertically and horizontally, but the factor of the upconversion process does not particularly matter and may be changeable to a desired factor.

The first upconverted image 56a is generated from the first debayered image 54a. The first up-converted image 56a further includes RGB values corresponding to the positions (phases) of pixels different from the pixel provided with the first polarizer 24a. The first up-converted image 56a further includes, for example, RGB values RGB11b, RGB12b, RGB13b, and RGB14b corresponding to the positions (phases) of the pixels provided with the second polarizer 24b. The first up-converted image 56a further includes, for example, RGB values RGB11c, RGB12c, RGB13c, and RGB14c corresponding to the positions (phases) of the pixels provided with the third polarizer 24c. The first up-converted image 56a further includes, for example, RGB values RGB11d, RGB12d, RGB13d, and RGB14d corresponding to the positions (phases) of the pixels provided with the fourth polarizer 24d. Therefore, the first up-converted image 56a of FIG. 6 includes the RGB value of the first polarization component set for each pixel, the number of pixels being equal to the number of pixels in the input image 50.

The second upconverted image 56b is generated from the second debayered image 54b. The second up-converted image 56b further includes RGB values corresponding to the positions (phases) of pixels different from the pixel provided with the second polarizer 24b. The second up-converted image 56b further includes, for example, RGB values RGB21a, RGB22a, RGB23a, and RGB24a corresponding to the positions (phases) of the pixels provided with the first polarizer 24a. The second up-converted image 56b further includes, for example, RGB values RGB21c, RGB22c, RGB23c, and RGB24c corresponding to the positions (phases) of the pixels provided with the third polarizer 24c. The second up-converted image 56b further includes, for example, RGB values RGB21d, RGB22d, RGB23d, and RGB24d corresponding to the positions (phases) of the pixels provided with the fourth polarizer 24d. Therefore, the second up-converted image 56b of FIG. 6 includes the RGB value of the second polarization component set for each pixel, the number of pixels being equal to the number of pixels in the input image 50.

The third upconverted image 56c is generated from the third debayered image 54c. The third up-converted image 56c further includes RGB values corresponding to the positions (phases) of pixels different from the pixel provided with the third polarizer 24c. The third up-converted image 56c further includes, for example, RGB values RGB31a, RGB32a, RGB33a, and RGB34a corresponding to the positions (phases) of the pixels provided with the first polarizer 24a. The third up-converted image 56c further includes, for example, RGB values RGB31b, RGB32b, RGB33b, and RGB34b corresponding to the positions (phases) of the pixels provided with the second polarizer 24b. The third up-converted image 56c further includes, for example, RGB values RGB31d, RGB32d, RGB33d, and RGB34d corresponding to the positions (phases) of the pixels provided with the fourth polarizer 24d. Therefore, the third up-converted image 56c of FIG. 6 includes the RGB value of the third polarization component set for each pixel, the number of pixels being equal to the number of pixels in the input image 50.

The fourth upconverted image 56d is generated from the fourth debayered image 54d. The fourth up-converted image 56d further includes RGB values corresponding to the positions (phases) of pixels different from the pixel provided with the fourth polarizer 24d. The fourth up-converted image 56d further includes, for example, RGB values RGB41a, RGB42a, RGB43a, and RGB44a corresponding to the positions (phases) of the pixels provided with the first polarizer 24a. The fourth up-converted image 56d further includes, for example, RGB values RGB41b, RGB42b, RGB43b, and RGB44b corresponding to the positions (phases) of the pixels provided with the second polarizer 24b. The fourth up-converted image 56d further includes, for example, RGB values RGB41c, RGB42c, RGB43c, and RGB44c corresponding to the positions (phases) of the pixels provided with the third polarizer 24c. Therefore, the fourth up-converted image 56d of FIG. 6 includes the RGB value of the fourth polarization component set for each pixel, the number of pixels being equal to the number of pixels in the input image 50.

The interpolation processing unit 40 calculates, pixel by pixel, the RGB value of each of the four polarization components by generating the four upconverted images 56a-56d corresponding to the four polarization components in this way. For example, the RGB values of the four polarization components in the pixel corresponding to the pixel value R1 of the input image 50 of FIG. 6 are RGB11, RGB21a, RGB31a, and RGB41a.

Hereinafter, the RGB values of the four polarization components of each pixel output from the interpolation processing unit 40 are denoted by RGBa, RGBb, RGBc, and RGBd. RGBa denotes the RGB value of the first polarization component, RGBb denotes the RGB value of the second polarization component, RGBc denotes the RGB value of the third polarization component, and RGBd denotes the RGB value of the fourth polarization component. Given that the pixel corresponding to the pixel value R1 of the input image 50 of FIG. 6 is a pixel of interest, RGBa=RGB11, RGBb=RGB21a, RGBc=RGB31a, RGBd=RGB41a.

Referring back to FIG. 1, the brightness calculation unit 42 calculates, pixel by pixel, the brightness value (Y value) of each of the four polarization components from the RGB values (RGBa to RGBd) of the four polarization components, respectively. For example, the brightness calculation unit 42 can calculate the Y value from the RGB value by using the following expression (1) provided by the international standard (ITU-R BT.709) that defines a video signal of the HDTV broadcasting system.

Y=0.2126R+0.7152G+0.722B (1)

The brightness calculation unit 42 calculates, pixel by pixel, the brightness value Ya of the first polarization component, the brightness value Yb of the second polarization component, the brightness value Yc of the third polarization component, and the brightness value Yd of the fourth polarization component.

The rank processing unit 44 ranks, pixel by pixel, the four polarization components in the order of the magnitude of the brightness values Ya to Yd of the four polarization components. The rank processing unit 44 outputs rank signals D1 to D4 indicating the polarization components of the 1st to 4th place. For example, the rank processing unit 44 outputs a first-place signal D1 indicating a polarization component with the maximum brightness value, a second-place signal D2 indicating a polarization component with the next largest brightness value, a third-place signal D3 indicating a polarization component with the next largest brightness value, and a fourth-place signal D4 indicating a polarization component with the minimum brightness value. Each of the rank signals D1 to D4 outputs, for example, one of values “0”, “1”, “2”, and “3” as an identification value (number) for distinguishing the four polarization components. For example, the first polarization component is identified by “0”, the second polarization component is identified by “1”, the third polarization component is identified by “2”, and the fourth polarization component is identified by “3”. Given, for example, that the magnitude of the brightness value in a particular pixel is such that Yc>Yb>Yd>Ya, the first-place signal D1=2, the second-place signal D2=1, the third-place signal D3=3, and the fourth-place signal D4=0. Since the order of the four polarization components may vary depending on the pixel, the output values of the four rank signals D1 to D4 may vary depending on the pixel.

The reference generation unit 46 calculates, pixel by pixel, a reference brightness value Ys obtained by synthesizing the brightness values of a plurality of polarization components. The reference brightness value Y s can be calculated by using the following expression (2).

Ys=k1·Y1+k2·Y2+k3·Y3+k4·Y4 (2)

The brightness values Y1 to Y4 are brightness values of the first-place to fourth-place polarization components ranked by the rank processing unit 44. The coefficients k1 to k4 are weight coefficients for synthesizing the brightness values Y1 to Y4. The coefficients k1 to k4 are set such that at least the second coefficient k2 and the third coefficient k3 are not 0. Stated otherwise, the reference brightness value Y s is calculated by synthesizing the brightness values Y2, Y3 of at least the second-place and third-place polarization components. Thereby, the reference brightness value Ys can represent an intermediate brightness value of the brightness values Y1 to Y4 of the four polarization components. For example, the four coefficients k1 to k4 can be such that k1=0, k2=0.5, k3=0.5, k4=0. In this case, the reference brightness value Y s is the average value of the brightness values Y2 and Y3 of the second-place and third-place polarization components. For example, the four coefficients k1 to k4 can be such that k1=0.25, k2=0.25, k3=0.25, k4=0.25. In this case, the reference brightness value Y s is the average value of the four polarization components Y1 to Y4.

The synthesis processing unit 48 calculates, pixel by pixel, a composite RGB value obtained by synthesizing the RGB values of a plurality of polarization components. The synthesis processing unit 48 changes the method for calculating the composite RGB value according to the magnitude of the reference brightness value Ys. When the reference brightness value Y s matches a predetermined threshold value Yth (i.e., Ys=Yth), the synthesis processing unit 48 defines the reference RGB value (RGBs) to be the composite RGB value (RGBm) (i.e., RGBm=RGBs). The reference RGB value (RGBs) is a value obtained by subjecting the RGB values of the four polarization components to weighted averaging by using the coefficients k1 to k4 and can be calculated by using the following expression (3).

RGBs=k1·RGB1+k2·RGB2+k3·RGB3+k4·RGB4 (3)

The values of the coefficients k1 to k4 are the same as the values used when the reference brightness value Ys is calculated by the reference generation unit 46. For example, k1=0, k2=0.5, k3=0.5, k4=0. Further, RGB1 to RGB4 are the RGB values of the first-place to fourth-place polarization components ranked by the rank processing unit 44. The threshold value Yth is set to be a brightness value that gives an appearance intermediate in brightness between black (e.g., minimum brightness value Ymin) and white (e.g., maximum brightness value Ymax). For example, the threshold value Yth is set to a value 18% of the maximum brightness value Y max. In the case that the brightness value comprises 12 bits, Ymax=4095 and Yth=737.

When the reference brightness value Ys is smaller than the predetermined threshold value Yth (i.e., Ys<Yth), the synthesis processing unit 48 calculates the composite RGB value obtained by synthesizing a high-rank RGB value (RGBt), derived from synthesizing the RGB values of the first-place and second-place polarization components, and the reference RGB value (RGBs). When the reference brightness value Ys is smaller than the threshold value Yth, the composite RGB value can be ensured to be greater than the reference RGB value by synthesizing the high-rank RGB value greater than the reference RGB value. Thereby, underexposure in dark pixels can be suppressed.

The high-rank RGB value (RGBt) is a weighted average value of the RGB value (RGB1) of the first-place polarization component and the RGB value (RGB2) of the second-place polarization component. The synthesis processing unit 48 can, for example, calculate the high-rank RGB value (RGBt) pixel by pixel by using the following expression (4).

RGBt=(RGB1·RGB1+RGB2·RGB2)/(RGB1+RGB2) (4)

In the above expression (4), weighted averaging is carried out based on the magnitude of the brightness of RGB1 and RGB2 so that the RGB value (RGB1) of the first-place polarization component is given a greater weight in the synthesis.

In the case that Ys<Yth, the synthesis processing unit 48 can calculate the composite RGB value (RGBm) pixel by pixel by using the following expression (5).

RGBm=t·RGBt+(1−t)·RGBs (5)

The high-rank weight coefficient t can be calculated pixel by pixel by using the following expression (6).

t=(Yth−Ys)/Yth (6)

The high-rank weight coefficient t is set to increase as the reference brightness value Y s decreases according to the above expression (6). For example, when Ys=Yth, t=0, and the above expression (5) will be RGBm=RGBs. When Ys=0, t=1, and the above expression (5) will be RGBm=RGBt. In the case that Ys<Yth, therefore, the smaller the reference brightness value Ys (i.e., as the darkness increases), the greater the weight of the high-rank RGB value (RGBt) in the composite RGB value (RGBm) and the smaller the weight of the reference RGB value (RGBs).

When the reference brightness value Ys is greater than the predetermined threshold value Yth (i.e., Ys>Yth), the synthesis processing unit 48 calculates the composite RGB value obtained by synthesizing a low-rank RGB value (RGBu), derived from synthesizing the RGB values of the third-place and fourth-place polarization components, and the reference RGB value (RGBs). When the reference brightness value Ys is greater than the threshold value Yth, the composite RGB value can be ensured to be smaller than the reference RGB value by synthesizing the low-rank RGB value including a value smaller than the reference RGB value. Thereby, overexposure in bright pixels can be suppressed.

The low-rank RGB value (RGBu) is a weighted average value of the RGB value (RGB3) of the third-place polarization component and the RGB value (RGB4) of the fourth-place polarization component. The synthesis processing unit 48 can, for example, calculate the low-rank RGB value (RGBu) pixel by pixel by using the following expression (7).

RGBu=[RGB4·(RGBmax−RGB4)+RGB3·(RGBmax−RGB3)]/[(RGBmax−RGB4)+(RGBmax−RGB3)] (7)

where RGBmax denotes the maximum RGB value of the pixel. In the case that the brightness value comprises 12 bits, RGBmax=4095. In the above expression (7), the value obtained by subtracting the RGB value from the maximum pixel value (RGBmax) is used as the weight coefficient. The value obtained by subtracting the RGB value from the maximum pixel value (RGBmax) represents the magnitude of the darkness of the RGB value. In the above expression (7), weighted averaging is carried out based on the magnitude of the darkness of RGB4 and RGB3 so that the RGB value (RGB4) of the fourth-place polarization component is given a greater weight in the synthesis.

In the case that Ys>Yth, the synthesis processing unit 48 can calculate the composite RGB value (RGBm) pixel by pixel by using the following expression (8).

RGBm=u·RGBu+(1−u)·RGBs (8)

where the low-rank weight coefficient u can be calculated pixel by pixel by using the following expression (9)

u=(Ys−Yth)/(Ymax−Yth) (9)

The low-rank weight coefficient u is set to increase as the reference brightness value Y s increases according to the above expression (9). For example, when Ys=Yth, u=0, and the above expression (8) will be RGBm=RGBs. When Ys=Ymax, u=1, and the above expression (8) will be RGBm=RGBu. In the case that Ys>Yth, therefore, the greater the reference brightness value Ys (i.e., as the brightness increases), the greater the weight of the low-rank RGB value (RGBu) in the composite RGB value (RGBm) and the smaller the weight of the reference RGB value (RGBs).

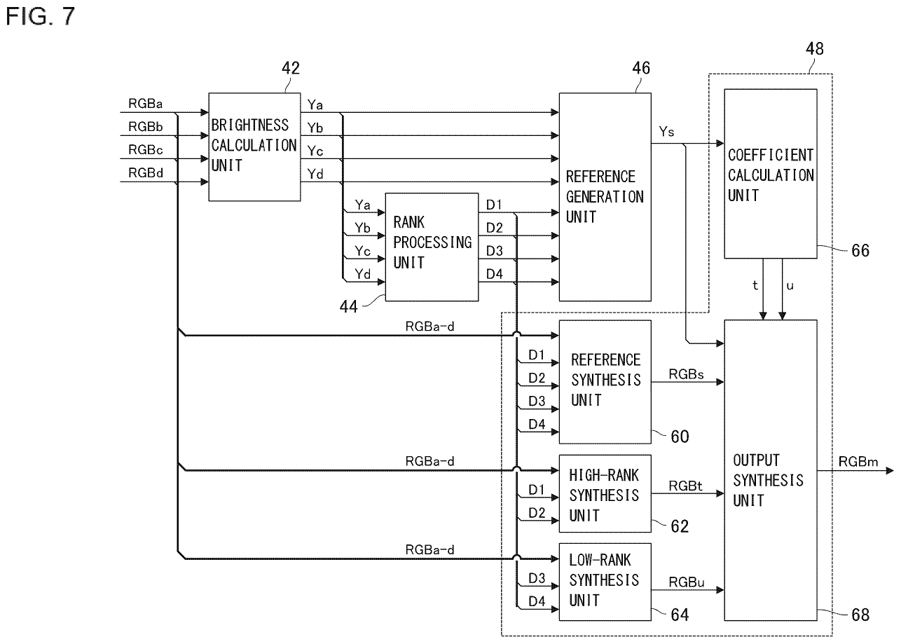

FIG. 7 schematically shows the flow of the image process performed by the synthesis processing unit 48. FIG. 7 shows the flow of the process of receiving the RGB values (RGBa to RGBd) of the four polarization components output from the interpolation processing unit 40 as inputs and the outputting the composite RGB values (RGBm). FIG. 7 also shows the brightness calculation unit 42, the rank processing unit 44, and the reference generation unit 46.

The brightness calculation unit 42 uses the RGB values (RGBa to RGBd) of the four polarization components as inputs and outputs the brightness values (Ya to Yd) of the four polarization components. The brightness calculation unit 42 calculates the brightness values (Ya to Yd) from the RGB values (RGBa to RGBd) by using the above expression (1).

The rank processing unit 44 uses the brightness values (Ya to Yd) output from the brightness calculation unit 42 as inputs, ranks the magnitude of the brightness values, and outputs rank signals D1 to D4 indicating the order of the four polarization components.

The reference generation unit 46 outputs the reference brightness value Ys by using brightness values (Ya to Yd) output from the brightness calculation unit 42 and the rank signals (D1 to D4) output from the rank processing unit 44 as inputs. The reference generation unit 46 calculates the reference brightness value Ys by using the above expression (2). The reference generation unit 46 can use, for example, the values of the coefficients k1 to k4 specified by a register (not shown).

The synthesis processing unit 48 includes a reference synthesis unit 60, a high-rank synthesis unit 62, a low-rank synthesis unit 64, a coefficient calculation unit 66, and an output synthesis unit 68.

The reference synthesis unit 60 calculates the reference RGB value (RGBs) by using the RGB values (RGBa to RGBd) of the four polarization components output from the interpolation processing unit 40 and the rank signals (D1 to D4) output from the rank processing unit 44 as inputs. The reference synthesis unit 60 calculates reference RGB value (RGBs) by using the above expression (3). The reference synthesis unit 60 defines the RGB value of the polarization component specified by the value of the first-place signal D1 to be RGB1, defines the RGB value of the polarization component specified by the value of the second-place signal D2 to be RGB2, defines the RGB value of the polarization component specified by the value of the third-place signal D3 to be RGB3, and defines the RGB value of the polarization component specified by the value of the fourth-place signal D4 to be RGB4. The reference synthesis unit 60 can, for example, use the values of the coefficients k1 to k4 specified by a register (not shown) common to the reference synthesis unit 60 and the brightness calculation unit 42.

The high-rank synthesis unit 62 calculates the high-rank RGB value (RGBt) by using the RGB values (RGBa to RGBd) of the four polarization components output from the interpolation processing unit 40 and the first-place signal D1 and the second-place signal D2 output from the rank processing unit 44 as inputs. The high-rank synthesis unit 62 defines the RGB value of the polarization component specified by the value of the first-place signal D1 to be RGB1, defines the RGB value of the polarization component specified by the value of the second-place signal D2 to be RGB2, and calculates the high-rank RGB value (RGBt) by using the above expression (4).

The low-rank synthesis unit 64 calculates the low-rank RGB value (RGBu) by using the RGB values (RGBa to RGBd) of the four polarization components output from the interpolation processing unit 40 and the third-place signal D3 and the fourth-place signal D4 output from the rank processing unit 44 as inputs. The low-rank synthesis unit 64 defines the RGB value of the polarization component specified by the value of the third-place signal D3 to be RGB3, defines the RGB value of the polarization component specified by the value of the fourth-place signal D4 to be RGB4, and calculates the low-rank RGB value (RGBu) by using the above expression (7).

The coefficient calculation unit 66 calculates the high-rank weight coefficient t and the low-rank weight coefficient u by using the reference brightness value Ys output from the reference synthesis unit 60 as an input. The coefficient calculation unit 66 calculates the high-rank weight coefficient t by using the above expression (6). The coefficient calculation unit 66 calculates the low-rank weight coefficient u by using the above expression (9). The coefficient calculation unit 66 can, for example, use the value of Yth specified by a register (not shown).

The output synthesis unit 68 calculates the composite RGB value (RGBm) by using the reference brightness value Ys output from the reference generation unit 46, the reference RGB value (RGBs) output from the reference synthesis unit 60, the high-rank RGB value (RGBt) output from the high-rank synthesis unit 62, the low-rank RGB value (RGBu) output from the low-rank synthesis unit 64, and the high-rank weight coefficient t and the low-rank weight coefficient u output from the coefficient calculation unit 66 as inputs. The output synthesis unit 68 defines the composite RGB value (RGBm) to be equal to the reference RGB value (RGBs) in the case that a) Ys=Yth. In the case that b) Ys<Yth, the output synthesis unit 68 calculates the composite RGB value (RGBm) by synthesizing the high-rank RGB value (RGBt) and the reference RGB value (RGBs) by using the above expression (5). In the case that c) Ys>Yth, the output synthesis unit 68 calculates the composite RGB value (RGBm) by synthesizing the low-rank RGB value (RGBu) and the reference RGB value (RGBs) by using the above expression (8).

For example, the output synthesis unit 68 sequentially calculates the composite RGB value (RGBm) for each of the pixels to be processed. Thereby, the output composition unit 68 can generate a color image in which the RGB value of each pixel is the composite RGB value (RGBm).

According to this embodiment, a color image in which overexposure and underexposure are suppressed can be generated by synthesizing the RGB values of the four polarization components acquired by using the polarization image sensor. When a normal (non-polarization) image sensor that does not use a polarizer is used, a color image affected by overexposure or underexposure may be generated depending on how the light is reflected by the subject. When it is attempted to photograph a subject through a glass, for example, the light strongly reflected by the glass surface may cause overexposure, resulting in an image that cannot properly capture the subject beyond the glass. Further, in the case of a subject exposed to strong light, an image affected by underexposure in the shadow of the subject may result. According to this embodiment, the influence of the highest ranking (i.e., the first-place) polarization component that can cause overexposure can be suppressed, and the influence of the lowest ranking (i.e., the fourth-place) polarization component that can cause underexposure can be suppressed, by using the reference RGB value (RGBs) obtained by synthesizing the RGB values of a plurality of polarization components including at least the second-place and third-place polarization components as the basis of the pixel value.

According to this embodiment, the pixel value of a pixel with a relatively great reference brightness value Ys can be decreased as compared to the case of employing the reference RGB value, by increasing the contribution of the low-rank RGB value (RGBu) to calculate the composite RGB value (RGBm. Thereby, the occurrence of overexposure can be suitably suppressed. For example, the occurrence of overexposure can be more suitably suppressed by increasing the weight of the low-rank RGB value (RGBu) in the synthesis as the reference brightness value Ys increases. Further, the pixel value of a pixel with a relatively small reference brightness value Ys can be increased as compared to the case of employing the reference RGB value, by increasing the contribution of the high-rank RGB value (RGBt) to calculate the composite RGB value (RGBm). Thereby, the occurrence of underexposure can be suitably suppressed. For example, the occurrence of underexposure can be more suitably suppressed by increasing the weight of the high-rank RGB value (RGBt) in the synthesis as the reference brightness value Ys decreases.

### Second Embodiment

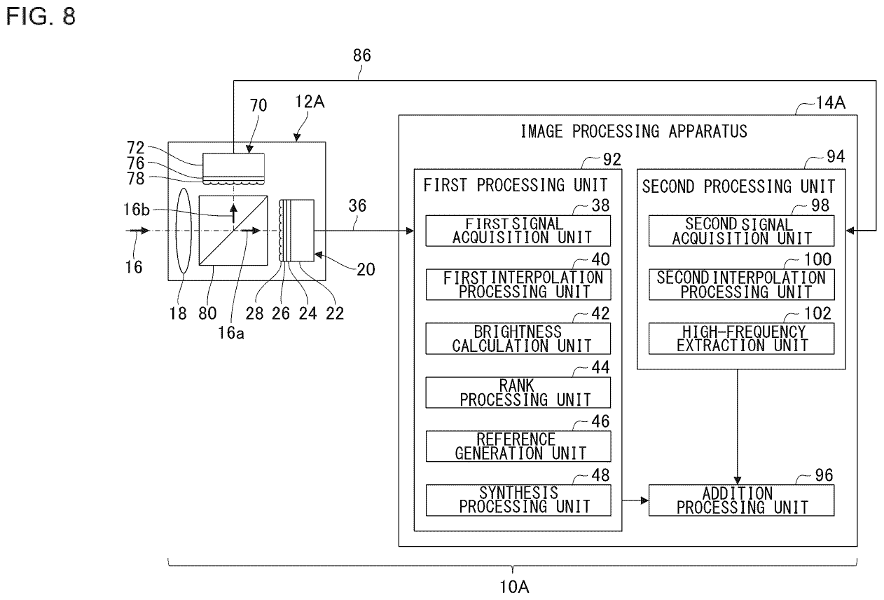

FIG. 8 schematically shows a configuration of an imaging apparatus 10A according to the second embodiment. The imaging apparatus 10A according to the second embodiment includes an imaging unit 12A and an image processing apparatus 14A. The following description of the second embodiment highlights the difference from the first embodiment. A description of common features is omitted as appropriate. The features identical to those of the first embodiment are denoted by identical reference symbols in the drawings.

The imaging unit 12A includes an imaging lens 18, a polarization image sensor 20, a non-polarization image sensor 70, and a light splitting element 80. The imaging lens 18 and the polarization image sensor 20 are configured in the same manner as in the first embodiment. The imaging unit 12A differs from that of the first embodiment in that the imaging unit 12A further includes the non-polarization image sensor 70 and the light splitting element 80.

The light splitting element 80 is arranged behind the imaging lens 18. The light splitting element 80 splits the incident light 16 that has passed through the imaging lens 18 into a first light 16a and a second light 16b. The first light 16a is incident on the polarization image sensor 20, and the second light 16b is incident on the non-polarization image sensor 70. The light splitting element 80 is, for example, a non-polarization light beam splitter and splits the incident light 16 into the first light 16a and the second light 16b in an 1:1 intensity ratio. The partial reflecting surface of the light splitting element 80 is composed of, for example, a half mirror made of a metal thin film, etc.

The polarization image sensor 20 images the first light 16a split by the light splitting element 80. The non-polarization image sensor 70 images the second light 16b split by the light splitting element 80. The polarization image sensor 20 and the non-polarization image sensor 70 are arranged so as to be coaxial with reference to the optical axis of the incident light 16. The imaging lens 18 is arranged so as to form an image of the incident light 16 on the light receiving surface of each of the polarization image sensor 20 and the non-polarization image sensor 70.

The non-polarization image sensor 70 includes a plurality of pixels for imaging the incident light 16. The non-polarization image sensor 70 includes a light detection layer 72, a color filter layer 76, and a microlens layer 78. The non-polarization image sensor 70 differs from the polarization image sensor 20 in that the non-polarization image sensor 70 does not include a polarizer layer.

The number of pixels of the non-polarization image sensor 70 is greater than the number of arrangements of the pixel groups 32 of the polarization image sensor 20. Therefore, the number of pixels in the height and width of the non-polarization image sensor 70 is greater than ½ of the number of pixels in the height and width of the polarization image sensor 20. The number of pixels in the height and width of the non-polarization image sensor 70 may be the same as the number of pixels in the height and width of the polarization image sensor 20. The number of pixels in the height and width of the non-polarization image sensor 70 may be greater than the number of pixels in the height and width of the polarization image sensor 20. For example, the number of pixels in the height and width of the non-polarization image sensor 70 may be twice or four times the number of pixels in the vertical and horizontal of the polarization image sensor 20.

The light detection layer 72 is, for example, configured in the same way as a two-dimensional image sensor such as a CCD (Charge Coupled Devices) sensor and a CMOS (Complementary Metal Oxide Semiconductor) sensor. The light detection layer 72 can be configured in the same manner as the light detection layer 22 of the polarization image sensor 20. The light detection layer 72 includes, for example, one photodiode for each pixel of the non-polarization image sensor 70. When the number of pixels of the non-polarization image sensor 70 and the number of pixels of the polarization image sensor 20 match, the light detection layer 72 may have the same specification as the light detection layer 22 of the polarization image sensor 20.

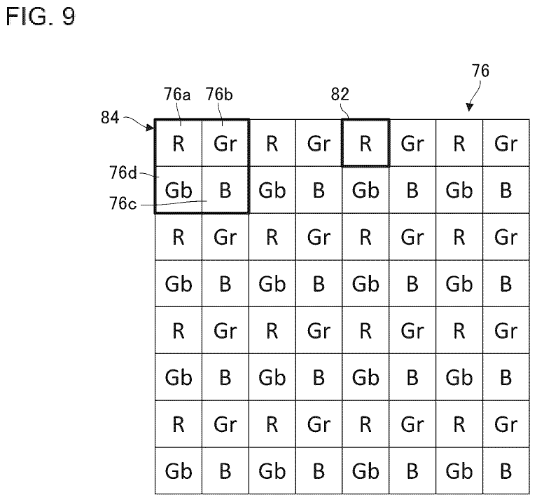

FIG. 9 is a plan view schematically showing a configuration of the color filter layer 76 of the non-polarization image sensor 70. The color filter layer 76 includes a red (R) filter 76a, a green (Gr) filter 76b, a blue (B) filter 76c, or a green (Gb) filter 76d in each pixel 82 of the non-polarization image sensor 70, the RGB filters being in the Bayer arrangement. The color filter layer 76 has a structure in which pixel groups 84 each including four pixels of 2×2 in the height and width are in a two-dimensional arrangement as repeating units.

Referring back to FIG. 8, the microlens layer 28 includes a plurality of microlenses in a two-dimensional arrangement. The microlens layer 28 can be configured in the same manner as the microlens layer 28 of the polarization image sensor 20. The microlens layer 78 includes, for example, one microlens for one pixel 82 of the non-polarization image sensor 70.

The image processing apparatus 14A generates an image by using an output signal of the polarization image sensor 20 and an output signal of the non-polarization image sensor 70. The image processing apparatus 14A includes a first processing unit 92, a second processing unit 94, and an addition processing unit 96.

The first processing unit 92 executes a signal process or an image process by using the first image signal 36 output from the polarization image sensor 20 as an input. The first processing unit 92 includes a first signal acquisition unit 38, a first interpolation processing unit 40, a brightness calculation unit 42, a rank processing unit 44, a reference generation unit 46, and a synthesis processing unit 48. The same function as that of the image processing apparatus 14 according to the first embodiment is realized.

The first processing unit 92 outputs a composite RGB value (RGBm) obtained by synthesizing the RGB values of the four polarization components to the addition processing unit 96. The first processing unit 92 may output the RGB values (RGBa to RGBd) of the four polarization components to the addition processing unit 96 instead of the composite RGB value (RGBm). The first processing unit 92 may output the composite RGB value (RGBm) and the RGB values (RGBa to RGBd) of the four polarization components to the addition processing unit 96.

The second processing unit 94 executes a signal process or an image process that uses a second image signal 86 output from the non-polarization image sensor 70 as an input. The second processing unit 94 includes a second signal acquisition unit 98, a second interpolation processing unit 100, and a high-frequency extraction unit 102.

The second signal acquisition unit 98 acquires the second image signal 86 output from the non-polarization image sensor 70. The second image signal 86 corresponds to raw data output from the non-polarization image sensor 70. For example, the second image signal 86 is serial data for the pixel value of each pixel 82 read in the order of address of each pixel 82 of the non-polarization image sensor 70. The second image signal 86 may not be strict RAW data but data derived from subjecting the RAW data to a correction process such as white balance adjustment and gain adjustment. The number of bits of the pixel value of the second image signal 86 does not particularly matter. For example, the pixel value includes 12 bits.

The second interpolation processing unit 100 generates one color image from the second image signal 86 acquired by the second signal acquisition unit 98. The second interpolation processing unit 100 calculates an RGB value set for each pixel 82 of the non-polarization image sensor 70 by executing a known debayering process.

The second interpolation processing unit 100 may resize the debayered image generated by the debayering process when the number of pixels of the non-polarization image sensor 70 is different from the number of pixels of the polarization image sensor 20. Thereby, the second interpolation processing unit 100 generates a color image including the same number of pixels in the height and width as the output image of the first processing unit 92.

The high-frequency extraction unit 102 extracts the high-frequency component of the RGB value of each pixel of the color image output from the second interpolation processing unit 100 and generates a high-frequency color image. The RGB value of each pixel of the high-frequency color image is array data comprising the pixel values (Rh, Gh, Bh) of the high-frequency component (Rh) of the R value, the high-frequency component (Gh) of the G value, and the high-frequency component (Bh) of the B value.

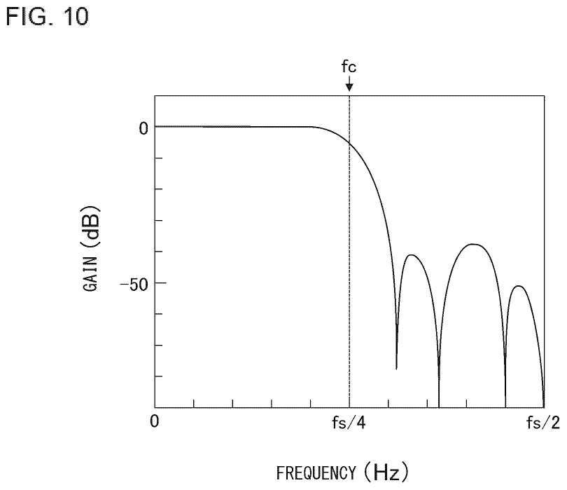

The high-frequency extraction unit 102 can calculate the high-frequency component by extracting the low-frequency component from the color image output from the second interpolation processing unit 100 and subtracts the low-frequency component from the original color image. For example, an FIR (Finite Impulse Response) filter, in which the cut-off frequency fc is ¼ of the sampling frequency fs (i.e., fc=fs/4), can be used as a low-pass filter (LPF) for extracting the low-frequency component. FIG. 10 shows an example of the frequency characteristics of the low-pass filter used in the high-frequency extraction unit 102. The LPF used in the high-frequency extraction unit 102 is not limited to the one shown in FIG. 10, and the cut-off frequency fc may be smaller or larger than ¼ of the sampling frequency fs.

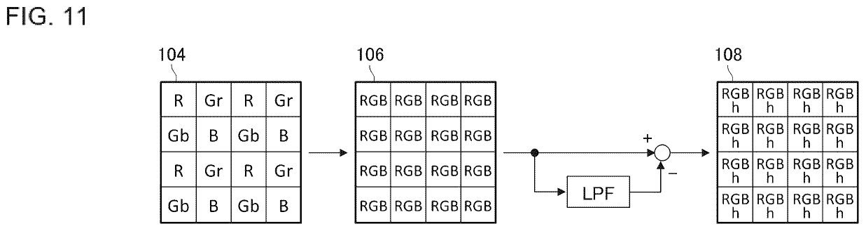

FIG. 11 schematically shows the flow of the image process performed by the second processing unit 94. FIG. 11 shows a clip of only 16 pixels of 4×4 in the height and width in order to simplify the explanation. An input image 104 corresponds to RAW data based on the second image signal 86, and only one pixel value is stored in each pixel. The input image 104 is a Bayer image corresponding to the arrangement of the color filter layer 76.

The second interpolation processing unit 100 generates a debayered image 106 from the input image 104. The debayered image 106 is a color image in which RGB values are stored in each pixel. In the example of FIG. 11, the process of resizing the debayered image 106 is omitted, but the second interpolation processing unit 100 may generate a color image obtained by resizing the debayered image 106.

The high-frequency extraction unit 102 generates a high-frequency color image 108 from the color image output from the second interpolation processing unit 100. The high-frequency color image 108 is a color image in which high-frequency RGB values (RGBh) indicating high-frequency components of the RGB values are stored in each pixel.

Referring back to FIG. 8, the addition processing unit 96 generates a high-resolution color image by adding, pixel by pixel, the composite RGB value (RGBm) output from the synthesis processing unit 48 of the first processing unit 92 and the high-frequency RGB value (RGBh) output from the second processing unit 94. The RGB value (RGBhr) of each pixel of the high-resolution color image is obtained by adding the high-frequency RGB value (RGBh) to the composite RGB value (RGBm), and RGBhr=RGBm+RGBh. The addition processing unit 96 may calculate a high-resolution RGB value (RGBhr) according to the expression RGBhr=RGBm+h·RGBh by using the weight coefficient h.

The addition processing unit 96 may generate a high-resolution color image by adding, pixel by pixel, the RGB values (RGBa to RGBd) of the four polarization components output from the first interpolation processing unit 40 of the first processing unit 92 and the high-frequency RGB value (RGBh) output from the second processing unit 94. The addition processing unit 96 may calculate the high-resolution RGB value (RGBhra=RGBa+RGBh) of the first polarization component by adding the high-frequency RGB value (RGBh) to the RGB value (RGBa) of the first polarization component. The addition processing unit 96 may calculate the high-resolution RGB value (RGBhrb=RGBb+RGBh) of the second polarization component by adding the high-frequency RGB value (RGBh) to the RGB value (RGBb) of the second polarization component. The addition processing unit 96 may calculate the high-resolution RGB value (RGBhrc=RGBc+RGBh) of the third polarization component by adding the high-frequency RGB value (RGBh) to the RGB value (RGBc) of the third polarization component. The addition processing unit 96 may calculate the high-resolution RGB value (RGBhrd=RGBd+RGBh) of the fourth polarization component by adding the high-frequency RGB value (RGBh) to the RGB value (RGBd) of the fourth polarization component.

According to this embodiment, a high-resolution color image can be generated by adding a high-frequency component obtained from the output signal of the non-polarization image sensor 70 to a color image obtained from the output signal of the polarization image sensor 20. As a result, a high-resolution color image with suppressed aliasing distortion (aliasing) can be obtained without using an optical low-pass filter.

Since the polarization image sensor 20 uses the pixel set 34 including 4×4 pixels as a repeating unit, the resolution tends to be lower than in the case of using an ordinary (non-polarization) image sensor that uses 2×2 pixels as a repeating unit. Further, when an optical low-pass filter is applied to the polarization image sensor 20 for the purpose of suppressing aliasing distortion, the polarization state changes due to the optical low-pass filter, which makes it impossible to detect a proper polarization component. Thus, an optical low-pass filter cannot be employed when the polarization image sensor 20 is used, and this can result in an image in which aliasing distortion is easily noticeable.

According to this embodiment, the non-polarization image sensor 70 not provided with a polarizer layer is used to make it easier to obtain a high-resolution image as compared to the polarization image sensor 20 and to extract a high-frequency component contributing to a high resolution. By adding such a high-frequency component to an image obtained by using the polarization image sensor 20, a color image with a high resolution and suppressed aliasing distortion can be generated.

According to this embodiment, as in the first embodiment, a color image in which overexposure and underexposure are suppressed can be generated. By adding a high-frequency component (high-frequency RGB value, RGBh) to a color image (composite RGB value, RGBm) in which overexposure and underexposure are suppressed, a more suitable color image (high-resolution RGB value, RGBhr) with a high resolution, suppressed aliasing distortion, and suppressed overexposure and underexposure can be generated.

According to this embodiment, by adding a high-frequency component (RGBh) to a color image (RGBa to RGBd) of the four polarization components, a more suitable polarized color image (RGBhra to RGBhrd) with a high resolution and suppressed aliasing distortion can be generated.

The addition processing unit 96 according to the second embodiment may not calculate the high-resolution RGB value (RGBhr) derived by using the composite RGB value (RGBm) and may calculate only one of the high-resolution RGB values (RGBhra to RGBhrd) of the four polarization components. In this case, the first processing unit 92 may not include the brightness calculation unit 42, the rank processing unit 44, the reference generation unit 46, and the synthesis processing unit 48. The first processing unit 92 may include only the first signal acquisition unit 38 and the first interpolation processing unit 40.

### Third Embodiment

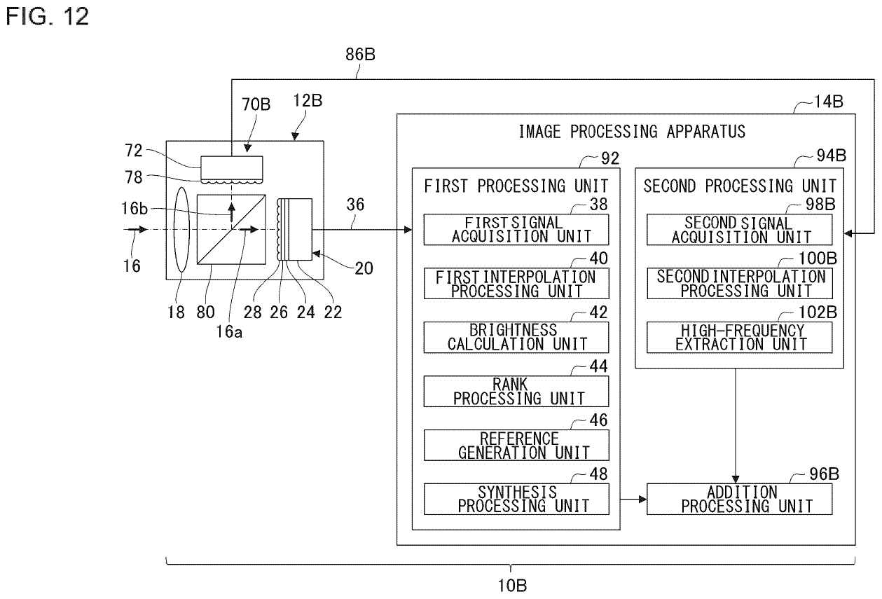

FIG. 12 schematically shows a configuration of an imaging apparatus 10B according to the third embodiment. The imaging apparatus 10B according to the third embodiment includes an imaging unit 12B and an image processing apparatus 14B. The following description of the third embodiment highlights the difference from the first embodiment or the second embodiment. A description of common features is omitted as appropriate. The features identical to those of the first embodiment or the second embodiment are denoted by identical reference symbols in the drawings.

The imaging unit 12B includes an imaging lens 18, a polarization image sensor 20, a non-polarization image sensor 70B, and a light splitting element 80. The imaging lens 18 and the polarization image sensor 20 are configured in the same manner as in the first embodiment. The light splitting element 80 is configured in the same manner as in the second embodiment.

The non-polarization image sensor 70B includes a light detection layer 72 and a microlens layer 78. The non-polarization image sensor 70B differs from the non-polarization image sensor 70 according to the second embodiment in that the non-polarization image sensor 70B does not include the color filter layer 76. Since the non-polarization image sensor 70B does not include the color filter layer 76, the non-polarization image sensor 70B functions as a non-polarization monochrome image sensor.

The image processing apparatus 14B includes a first processing unit 92, a second processing unit 94B, and an addition processing unit 96B. The first processing unit 92 is configured in the same manner as in the second embodiment. The second processing unit 94B includes a second signal acquisition unit 98B, a second interpolation processing unit 100B, and a high-frequency extraction unit 102B.

The second signal acquisition unit 98B acquires a second image signal 86B output from the non-polarization image sensor 70B. The second image signal 86B corresponds to raw data output from the non-polarization image sensor 70B. For example, the second image signal 86B is serial data for the pixel value of each pixel 82 read in the order of address of each pixel 82 of the non-polarization image sensor 70B. The second image signal 86B may not be strict RAW data but data derived from subjecting the RAW data to a correction process such as gain adjustment. The number of bits of the pixel value of the second image signal 86B does not particularly matter. For example, the pixel value includes 12 bits.

The second interpolation processing unit 100B generates one monochrome image from the second image signal 86B acquired by the second signal acquisition unit 98B. The second interpolation processing unit 100B outputs the brightness value (Y value) set for each pixel 82 of the non-polarization image sensor 70B. When the number of pixels of the non-polarization image sensor 70B is different from the number of pixels of the polarization image sensor 20, the second interpolation processing unit 100B resizes the monochrome image and generates a monochrome image in which the number of pixels in height and width match that of the output image of the first processing unit 92. When resizing is not necessary, the second processing unit 94B may not include the second interpolation processing unit 100B.

The high-frequency extraction unit 102B extracts the high-frequency component of the brightness value (Y value) of each pixel of the monochrome image output from the second signal acquisition unit 98B or the second interpolation processing unit 100B and generates a high-frequency monochrome image. The high-frequency extraction unit 102B can calculate the high-frequency component by extracting the low-frequency component from the monochrome image output from the second signal acquisition unit 98B or the second interpolation processing unit 100B and subtracting the low-frequency component from the original monochrome image. A filter similar to that of the second embodiment shown in FIG. 10 may be used as the LPF for extracting the low frequency component.

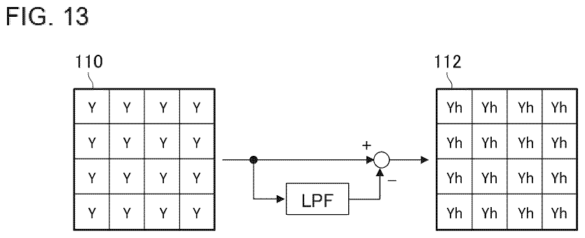

FIG. 13 schematically shows the flow of the image process performed by the second processing unit 94B. FIG. 13 shows a clip of only 16 pixels of 4×4 in the height and width in order to simplify the explanation. The input image 110 corresponds to the pixel array of RAW data based on the second image signal 86B, and only one pixel value is set in each pixel. The input image 110 is a monochrome image showing the brightness value (Y value) of each pixel.

The high-frequency extraction unit 102B generates a high-frequency monochrome image 112 from the monochrome image that is the input image 110. The high-frequency monochrome image 112 is a monochrome image in which a high-frequency brightness value (Yh) indicating the high-frequency component of the brightness value is set in each pixel.

Returning to FIG. 12, the addition processing unit 96B generates a high-resolution color image by adding, pixel by pixel, the composite RGB value (RGBm) output from the synthesis processing unit 48 of the first processing unit 92 and the high-frequency brightness value (Yh) output from the second processing unit 94B.

The RGB value (RGBhr) of each pixel of the high-resolution color image is obtained by adding the high-frequency brightness value (Yh) to the composite RGB value (RGBm), and RGBhr=RGBm+Yh. For example, the values (Rhr, Ghr, Bhr) of the respective colors of the high-resolution RGB value (RGBhr) are calculated by adding the common high-frequency brightness value Yh to each of the values (Rm, Gm, Bm) of the respective colors of the composite RGB value (RGBm). In other words, Rhr=Rm+Yh, Ghr=Gm+Yh, Bhr=Bm+Yh.

The addition processing unit 96B may calculate the high-resolution RGB value (RGBhr) according to the expression RGBhr=RGBm+h·RGBh by using the weight coefficient h. The weight coefficient h may be common to the respective colors or may vary depending on the color. In the latter case, the weight coefficients hR, hG, and hB of the respective colors may be used such that Rhr=Rm+hR·Yh, Ghr=Gm+hG·Yh, Bhr=Bm+hB·Yh. The weight coefficients hR, hG, and hB of the respective colors may be such that hR=0.2126, hG=0.7152, hB=0.0722 based on ITU-R BT.709.

The addition processing unit 96B may generate the high-resolution color image by adding, pixel by pixel, the RGB values (RGBa to RGBd) of the four polarization components output from the first interpolation processing unit 40 of the first processing unit 92 and the high-frequency brightness value (Yh) output from the second processing unit 94B. The addition processing unit 96B may calculate the high-resolution RGB value (RGBhra=RGBa+Yh) of the first polarization component by adding the high-frequency brightness value (Yh) to the RGB value (RGBa) of the first polarization component. The addition processing unit 96B may calculate the high-resolution RGB value (RGBhrb=RGBb+Yh) of the second polarization component by adding the high-frequency brightness value (Yh) to the RGB value (RGBb) of the second polarization component. The addition processing unit 96B may calculate the high-resolution RGB value (RGBhrc=RGBc+Yh) of the third polarization component by adding the high-frequency brightness value (Yh) to the RGB value (RGBc) of the third polarization component. The addition processing unit 96B may calculate the high-resolution RGB value (RGBhrd=RGBd+Yh) of the fourth polarization component by adding the high-frequency brightness value (Yh) to the RGB value (RGBd) of the fourth polarization component. When the addition processing unit 96B adds the high-frequency brightness value (Yh) to the RGB values (RGBa to RGBd) of the four polarization components, the weight coefficient h may be used, or the weight coefficients hR, hG, and hB of the respective color may be used.

According to this embodiment, the same advantage as that of the second embodiment can be achieved. According to this embodiment, the cost of the non-polarization image sensor 70B and the second processing unit 94B can be reduced because the high-frequency component obtained from the output signal of the non-polarized monochrome image sensor is added.

As in the second embodiment, the addition processing unit 96B according to the third embodiment may not calculate the high-resolution RGB value (RGBhr) derived by using the composite RGB value (RGBm) and may only calculate at least one of the high-resolution RGB values (RGBhra to RGBhrd) of the four polarization components. In this case, the first processing unit 92 may not include the brightness calculation unit 42, the rank processing unit 44, the reference generation unit 46, and the synthesis processing unit 48. The first processing unit 92 may include only the first signal acquisition unit 38 and the first interpolation processing unit 40.

### Fourth Embodiment

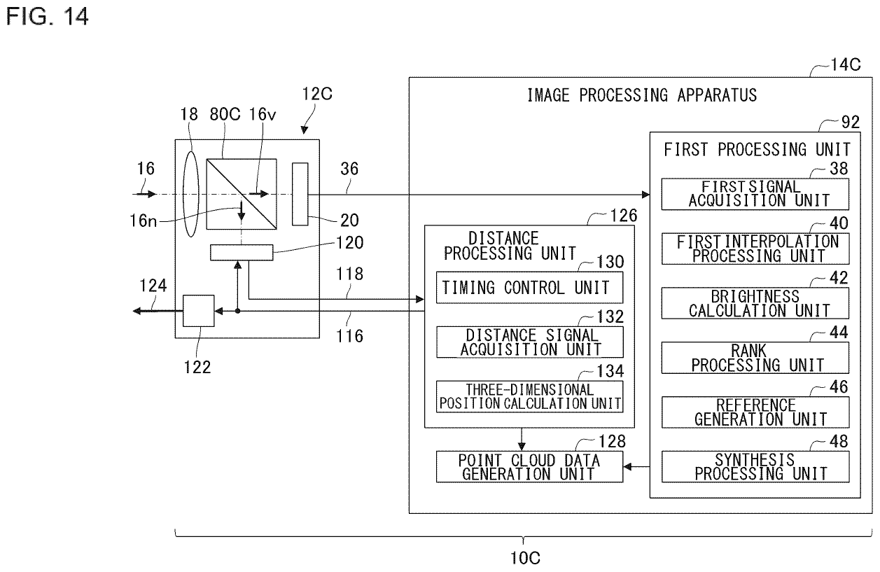

FIG. 14 schematically shows a configuration of an imaging apparatus 10C according to the fourth embodiment. The imaging apparatus 10C according to the fourth embodiment includes an imaging unit 12C and an image processing apparatus 14C. The following description of the fourth embodiment highlights the difference from the embodiments described above. A description of common features is omitted as appropriate. The features identical to those of the embodiments described above are denoted by identical reference symbols in the drawings.

The imaging unit 12C includes an imaging lens 18, a polarization image sensor 20, a light splitting element 80C, a distance image sensor 120, and an illumination apparatus 122. The imaging lens 18 and the polarization image sensor 20 are configured in the same manner as in the first embodiment.

The light splitting element 80C is arranged behind the imaging lens 18. The light splitting element 80C splits the incident light 16 that has passed through the imaging lens 18 into a visible light 16v and an infrared light 16n. The visible light 16v is incident on the polarization image sensor 20, and the infrared light 16n is incident on the distance image sensor 120. The light splitting element 80C is, for example, a dichroic prism and includes a dichroic mirror that selectively transmits visible light and selectively reflects infrared light. The dichroic mirror is composed of, for example, a dielectric multilayer film. The dichroic mirror is preferably a non-polarization dichroic mirror that is not polarization dependent. By employing a non-polarization dichroic mirror, it is possible to suppress a change in the polarization state of the visible light incident on the polarization image sensor 20. The light splitting element 80C may be configured to selectively reflect visible light and selectively transmit infrared light. In this case, the arrangement of the polarization image sensor 20 and the distance image sensor 120 is switched.

The polarization image sensor 20 and the distance image sensor 120 are arranged so as to be coaxial with reference to the optical axis of the incident light 16. The imaging lens 18 is arranged so as to form an image of the incident light 16 on the light receiving surface of each of the polarization image sensor 20 and the non-polarization image sensor 70.

The distance image sensor 120 detects the reflected light of an infrared illumination light 124 irradiating the subject from the illumination apparatus 122. The distance image sensor 120 includes a plurality of pixels provided with an infrared light filter that selectively transmits infrared light. The distance image sensor 120 is, for example, a LIDAR (Light Detection And Ranging) sensor and measures the distance to the subject according to the ToF (Time of Flight) scheme. The distance image sensor 120 outputs a distance value based on the time elapsed since the point of time of triggering of the pulse signal 116 supplied from the distance processing unit 126 until the point of time that the light is received in each pixel. The distance image sensor 120 outputs, for example, serial data for the distance value detected in each pixel as a distance image signal 118.

The illumination apparatus 122 radiates the infrared illumination light 124 toward the subject. The illumination apparatus 122 includes a laser diode array such as such as VCSEL (Vertical Cavity Surface Emitting Laser). The illumination apparatus 122 drives the laser diode array based on the pulse signal 116 supplied from the distance processing unit 126 and radiates a pulsed illumination light synchronized with the timing of triggering of the pulse signal 116.

The image processing apparatus 14C includes a first processing unit 92, a distance processing unit 126, and a point cloud data generation unit 128. The first processing unit 92 is configured in the same manner as in the second embodiment. The distance processing unit 126 includes a timing control unit 130, a distance signal acquisition unit 132, and a three-dimensional position calculation unit 134.

The timing control unit 130 generates a pulse signal 116 for driving the distance image sensor 120 and the illumination apparatus 122. The distance signal acquisition unit 132 acquires the distance image signal 118 from the distance image sensor 120. The three-dimensional position calculation unit 134 calculates coordinate values (x, y, z) indicating the three-dimensional position of the subject by using the distance image signal 118 as an input. The three-dimensional position calculation unit 134 calculates, pixel by pixel, coordinate values (x, y, z) indicating a three-dimensional position from the distance value detected in each pixel of the distance image sensor 120. The coordinate values (x, y, z) corresponding to each pixel correspond to the three-dimensional position of each point of reflection, on the subject surface, of the infrared light incident on each pixel.

The point cloud data generation unit 128 generates point cloud data that associates, pixel by pixel, the RGB value output from the first processing unit 92 with the coordinate value output from the distance processing unit 126. The point cloud data generation unit 128 can generate point cloud data that conforms to the PLY (Polygon File Format) file format. In the PLY file format, array data (x, y, z, R, G, B) is set for each pixel.

The point cloud data generation unit 128 may generate point cloud data that associates, pixel by pixel, the composite RGB value (RGBm) output from the synthesis processing unit 48 of the first processing unit 92 with the coordinate values (x, y, z) output from the distance processing unit 126. In this case, array data (x, y, z, Rm, Gm, Bm) is, for example, set for each pixel by using the PLY file format.

The point cloud data generation unit 128 may generate point cloud data that maps, pixel by pixel the RGB values (RGBa to RGBd) of the four polarization components output from the first interpolation processing unit 40 of the first processing unit 92 with the coordinate values (x, y, z) output from the distance processing unit 126. In this case, the point cloud data associates, pixel by pixel, the coordinate values (x, y, z), the RGB values (Ra, Ga, Ba) of the first polarization component, the RGB values (Rb, Gb, Bb) of the second polarization component, the RGB values (Rc, Gc, Bc) of the third polarization component, and the RGB values (Rd, Gd, Bd) of the fourth polarization component with each other. For example, array data (x, y, z, Ra, Ga, Ba, Rb, Gb, Bb, Rc, Gc, Bc, Rd, Gd, Bd) is set for each pixel by using the PLY file format. The order of setting the four polarization components may be the ascending order (e.g., 0 degrees, 45 degrees, 90 degrees, 135 degrees) or the descending order (e.g., 135 degrees, 90 degrees, 45 degrees, 0 degrees).

According to this embodiment, point cloud data that associates, pixel by pixel, the composite RGB value (RGBm) with the coordinate values (x, y, z) can be generated, and point cloud data with suppressed overexposure and underexposure can be provided. By modeling the three-dimensional shape of the subject in the virtual space based on such point cloud data, it is possible to provide a three-dimensional model that is not felt strange even when virtual illumination is applied to the subject in the virtual space.

According to this embodiment, point cloud data that maps, pixel by pixel, the RGB values (RGBa to RGBd) of the four polarization components with the coordinate values (x, y, z) can be generated. By using such point cloud data, it is possible to calculate a normal vector for each pixel from the four polarization components. Thereby, a three-dimensional model of the subject can be generated by using a normal vector and a point cloud of distance information for each pixel.

### Fifth Embodiment

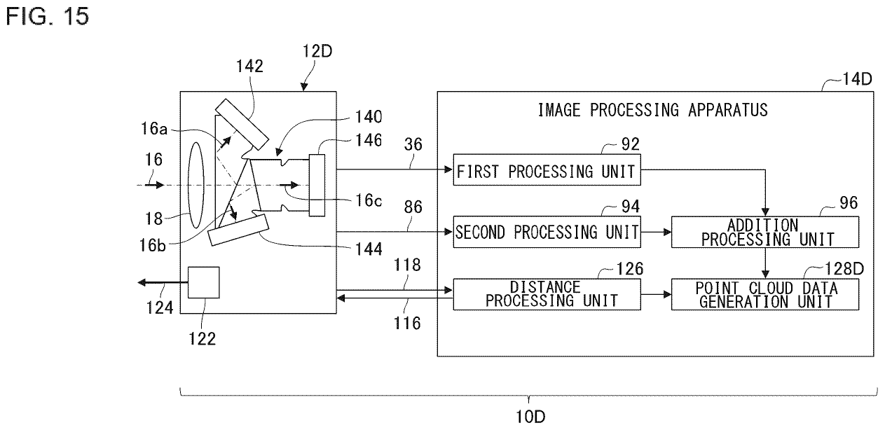

FIG. 15 schematically shows a configuration of an imaging apparatus 10C according to the fifth embodiment. The imaging apparatus 10D includes an imaging unit 12D and an image processing apparatus 14D. The following description of the fifth embodiment highlights the difference from the embodiments described above. A description of common features is omitted as appropriate. The features identical to those of the embodiments described above are denoted by identical reference symbols in the drawings.

The imaging unit 12D includes an imaging lens 18, a light splitting element 140, a first image sensor 142, a second image sensor 144, a third image sensor 146, and a illumination apparatus 122. The imaging unit 12D is a so-called three-plate camera, and the incident light 16 passing through the imaging lens 18 is split into three beams of light 16a, 16b, and 16c by using the light splitting element 140. The imaging unit 12D is configured to image the incident light 16 by each of the first image sensor 142, the second image sensor 144, and the third image sensor 146.

The light splitting element 140 is a so-called three-plate prism. The first image sensor 142, the second image sensor 144, and the third image sensor 146 are polarization image sensors, non-polarization image sensors, or distance image sensors. Specifically, one of the first image sensor 142, the second image sensor 144, and the third image sensor 146 is a polarization image sensor, and another one of the first image sensor 142, the second image sensor 144 and the third image sensor 146 is a non-polarization image sensor, and yet another one of the first image sensor 142, the second image sensor 144 and the third image sensor 146 is a distance image sensor.

The polarization image sensor included in the imaging unit 12D is configured in the same manner as the polarization image sensor 20 according to the above-described embodiment. The polarization image sensor outputs a first image signal 36. The non-polarization image sensor included in the imaging unit 12D is configured in the same manner as the non-polarization image sensor 70 according to the second embodiment described above or the non-polarization image sensor 70B according to the third embodiment. The non-polarization image sensor outputs a second image signal 86. The distance image sensor included in the imaging unit 12D is configured in the same manner as the distance image sensor 120 according to the fourth embodiment described above. The distance image sensor operates synchronously with the pulse signal 116 and outputs the distance image signal 118.

The illumination apparatus 122 is configured in the same manner as in the fourth embodiment described above. The illumination apparatus 122 operates synchronously with the pulse signal 116 and radiates the infrared illumination light 124 toward the subject.

The image processing apparatus 14D includes a first processing unit 92, a second processing unit 94, an addition processing unit 96, a distance processing unit 126, and a point cloud data generation unit 128D. The first processing unit 92, the second processing unit 94, and the addition processing unit 96 can be configured in the same manner as in the second or third embodiment described above. The distance processing unit 126 can be configured in the same manner as in the fourth embodiment described above.

The point cloud data generation unit 128D generates point cloud data that associates, pixel by pixel, the high-resolution RGB value (RGBhr or RGBhra to RGBhrd) output from the addition processing unit 96 with coordinate values (x, y, z) output from the distance processing unit 126.

The point cloud data generation unit 128D may generate point cloud data that associates the high-resolution RGB values (Rhr, Ghr, Bhr), derived by using the composite RGB value (RGBm), with the coordinate values (x, y, z). In this case, array data (x, y, z, Rhr, Ghr, Bhr) is set for each pixel by using, for example, the PLY file format.

The point cloud data generation unit 128D may generate point cloud data that associates, pixel by pixel, the high-resolution RGB values (RGBhra to RGBhrd), derived by using the RGB values (RGBa to RGBd) of the four polarization components, with the coordinate values (x, y, z). The point cloud data in this case associates, pixel by pixel, the coordinate values (x, y, z), the high-resolution RGB values (Rhra, Ghra, Bhra) of the first polarization component, the high-resolution RGB values (Rhrb, Ghrb, Bhrb) of the second polarization component, the high-resolution RGB values (Rhrc, Ghrc, Bhrc) of the third polarization component, and the high-resolution RGB values (Rhrd, Ghrd, Bhrd) of the fourth polarization component with each other. For example, array data (x, y, z, Rhra, Ghra, Bhra, Rhrb, Ghrb, Bhrb, Rhrc, Ghrc, Bhrc, Rhrd, Ghrd, Bhrd) is set for each pixel by using the PLY file format. The order of setting the four polarization components may be the ascending order (e.g., 0 degrees, 45 degrees, 90 degrees, 135 degrees) or the descending order (e.g., 135 degrees, 90 degrees, 45 degrees, 0 degrees).

According to this embodiment, point cloud data that associates, pixel by pixel, the high-resolution RGB value (RGBhr), derived by using the composite RGB value (RGBm), with the coordinate values (x, y, z) can be generated. Since the high-frequency component is added to the RGB value of the point cloud data, more suitable point cloud data with a high resolution, suppressed aliasing distortion, and suppressed overexposure and underexposure can be generated. By modeling the three-dimensional shape of the subject in the virtual space based on such point cloud data, it is possible to provide a three-dimensional model that is not felt strange even when virtual illumination is applied to the subject in the virtual space.

According to this embodiment, it is possible to generate point cloud data that associates, pixel by pixel, the high-resolution RGB values (RGBhra to RGBhrd), derived by using the RGB value (RGBa to RGBd) of the four polarization components, with the coordinate values (x, y, z). Since the high-frequency component is added to each of the RGB values of the four polarization components of the point cloud data, more suitable point cloud data with a high resolution and suppressed aliasing distortion can be provided. By using such point cloud data, it is possible to calculate a normal vector for each pixel from the four polarization components. Thereby, a three-dimensional model of the subject can be generated by using a normal vector and a point cloud of distance information for each pixel.

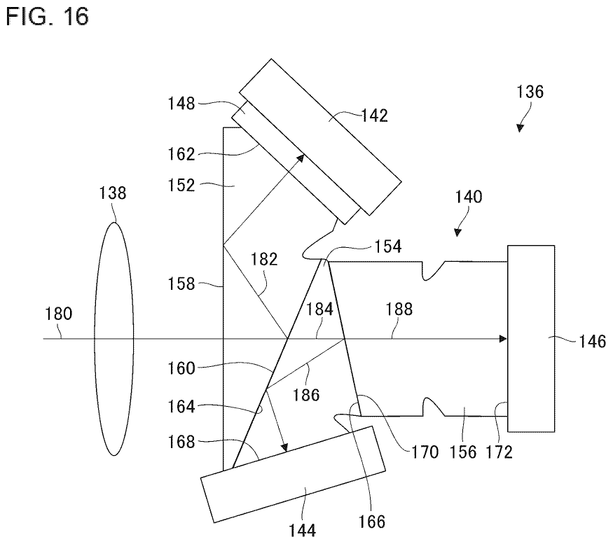

FIG. 16 schematically shows an imaging unit 136 according to the first exemplary configuration of the fifth embodiment. The imaging unit 136 of FIG. 16 can be used as the imaging unit 12D of FIG. 15. The imaging unit 136 includes an imaging lens 138, a light splitting element 140, a first image sensor 142, a second image sensor 144, a third image sensor 146, and a phase difference plate 148.

The light splitting element 140 includes a first prism 152, a second prism 154, and a third prism 156. The first prism 152 includes a first incidence surface 158, a first splitting surface 160, and a first exit surface 162. The second prism 154 includes a second incidence surface 164, a second splitting surface 166, and a second exit surface 168. The third prism 156 includes a third incidence surface 170 and a third exit surface 172. An air gap is provided between the first splitting surface 160 and the second incidence surface 164.

The incident light 180 incident on the first incidence surface 158 is split into a first reflected light 182 and a first transmitted light 184 at the first splitting surface 160. The first reflected light 182 reflected by the first splitting surface 160 is totally reflected internally by the first incidence surface 158, passes through the first exit surface 162, and travels toward the first image sensor 142. The first transmitted light 184 passing through the first splitting surface 160 is split into a second reflected light 186 and a second transmitted light 188 at the second splitting surface 166. The second reflected light 186 reflected by the second splitting surface 166 is totally reflected internally by the second incidence surface 164, passes through the second incidence surface 168, and travels toward the second image sensor 144. The second transmitted light 188 transmitted through the second splitting surface 166 passes through the third incidence surface 170 and the third exit surface 172 and travels toward the third image sensor 146.

In the first exemplary configuration of FIG. 16, the first image sensor 142 may be implemented by the polarization image sensor 20. The polarization state of the first reflected light 182 traveling toward the first image sensor 142 can be changed by being reflected by the first splitting surface 160 and the first incidence surface 158. In other words, the polarization state of the first reflected light 182 can change from the polarization state of the incident light 180. In particular, the polarization state of the first reflected light 182 can change considerably when the first reflected light 182 is totally reflected internally by the first incidence surface 158, which is the interface between the prism and the air. When the polarization state of the first reflected light 182 changes from the polarization state of the incident light 180, the polarization image sensor 20 cannot correctly measure the polarization state of the incident light 180.

In the first exemplary configuration of FIG. 16, the phase difference plate 148 is provided between the first image sensor 142 and the light splitting element 140 in order to compensate for the change in the polarization state of the first reflected light 182 caused by the light splitting element 140. The phase difference plate 148 is configured to provide a phase difference to reduce or cancel the phase difference between the s-polarization component and the p-polarization component of the first reflected light 182 produced by at least one of the reflection by the first splitting surface 160 or the reflection by the first incidence surface 158. The magnitude of the phase difference provided by the phase difference plate 148 does not particularly matter. For example, the phase difference is about 120 degrees.

When the first image sensor 142 is implemented by the polarization image sensor 20, the first splitting surface 160 preferably includes a non-polarization beam splitter that is not wavelength dependent. “Non-polarization” means that the impact on polarization is negligibly small. The non-polarization beam splitter is configured such that the change in the polarization state before and after reflection and before and after transmission in the beam splitter is negligible. A metal thin film can, for example, be used as a wavelength-independent non-polarization beam splitter. A dielectric multilayer designed to suppress a change in the polarization state can also be used as a wavelength-independent non-polarization beam splitter. By providing a non-polarization beam splitter on the first splitting surface 160, it is possible to suppress a change in the polarization state of the first reflected light 182 reflected by the first splitting surface 160.

When the first image sensor 142 is implemented by the polarization image sensor 20, one of the second image sensor 144 and the third image sensor 146 is the non-polarization image sensor 70 or 70B, and the other of the second image sensor 144 and the third image sensor 146 is the distance image sensor 120. In this case, the second splitting surface 166 preferably includes a dichroic mirror that isolates visible light and infrared light. A dielectric multilayer film can, for example, be used as a dichroic mirror. When the second image sensor 144 is implemented by the non-polarization image sensor 70 or 70B, the dichroic mirror provided on the second splitting surface 166 is designed to selectively reflect visible light and selectively transmit infrared light. When the second image sensor 144 is implemented by the distance image sensor 120, the dichroic mirror provided on the second splitting surface 166 is designed to selectively transmit visible light and selectively reflect infrared light. The second splitting surface 166 may include a wavelength-independent beam splitter such as a half mirror instead of a dichroic mirror.

According to the first exemplary configuration of FIG. 16, it is possible, by using the light splitting element 140, to image the incident light 180 to obtain a polarization image, a non-polarization image, and a distance image by using the polarization image sensor 20, the non-polarization image sensor 70 or 70B, and the distance image sensor 120, respectively. According to this embodiment, it is possible to provide a configuration in which a dichroic mirror is not arranged on the optical path toward the polarization image sensor 20 by implementing the first image sensor 142 by the polarization image sensor 20. Thereby, a polarization image that is not affected by a change in the polarization state in the dichroic mirror can be obtained. Further, it is possible to suppress a change in the polarization state of the first reflected light 182 produced in at least one of the first splitting surface 160 or the first incidence surface 158, by providing the phase difference plate 148 between the light splitting element 140 and the polarization image sensor 20 (first image sensor 142). Thereby, a polarization image in which the change in the polarization state with respect to the incident light 180 is suppressed can be obtained.

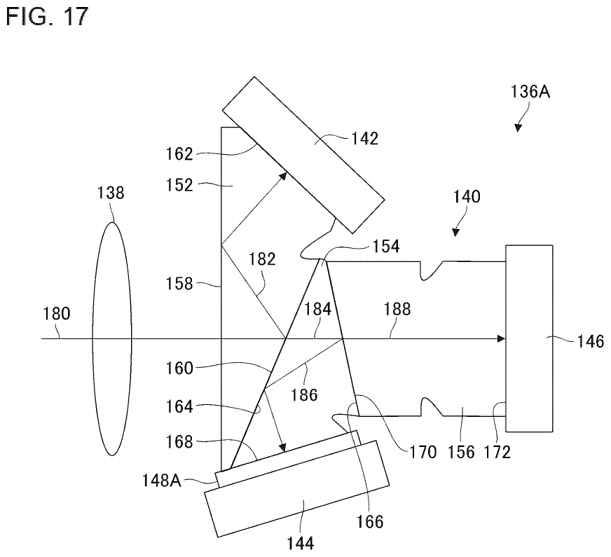

FIG. 17 schematically shows an imaging unit 136A according to the second exemplary configuration of the fifth embodiment. The second exemplary configuration differs from the first exemplary configuration described above in that a phase difference plate 148A is provided between the second image sensor 144 and the light splitting element 140. The following description of the second exemplary configuration highlights the difference from the first exemplary configuration. A description of common features is omitted as appropriate.

The imaging unit 136A includes an imaging lens 138, a light splitting element 140, a first image sensor 142, a second image sensor 144, a third image sensor 146, and a phase difference plate 148A. In the second exemplary configuration, the second image sensor 144 is implemented by the polarization image sensor 20. In the second exemplary configuration, one of the first image sensor 142 and the third image sensor 146 is implemented by the non-polarization image sensor 70 or 70B, and the other of the first image sensor 142 and the third image sensor 146 is implemented by the distance image sensor 120.

When the first image sensor 142 is implemented by the non-polarization image sensor 70 or 70B and the third image sensor 146 is implemented by the distance image sensor 120, the first splitting surface 160 preferably includes a wavelength-independent non-polarization beam splitter. By providing a non-polarization light beam splitter on the first splitting surface 160, it is possible to suppress a change in the polarization state of the first transmitted light 184 passing through the first splitting surface 160. When the first image sensor 142 is implemented by the non-polarization image sensor 70 or 70B and the third image sensor 146 is implemented by the distance image sensor 120, the second splitting surface 166 is preferably a non-polarization dichroic mirror. The dichroic mirror provided on the second splitting surface 166 is designed to selectively reflect visible light and selectively transmit infrared light. By providing a non-polarization dichroic mirror on the second splitting surface 166, it is possible to suppress a change in the polarization state of the second reflected light 186 reflected by the second splitting surface 166. The second splitting surface 166 may include a non-polarization beam splitter that is not wavelength-dependent such as a half mirror instead of a dichroic mirror.

When the first image sensor 142 is implemented by the distance image sensor 120 and the third image sensor 146 is implemented by the non-polarization image sensor 70 or 70B, the first splitting surface 160 is preferably a non-polarization dichroic mirror. A dielectric multilayer film designed to suppress a change in the polarization state from the visible range to the infrared region can, for example, be used as the non-polarized dichroic mirror. The dichroic mirror provided on the first splitting surface 160 is designed to selectively transmit visible light and selectively reflect infrared light. By providing a non-polarization dichroic mirror on the first splitting surface 160, it is possible to suppress a change in the polarization state of the first transmitted light 184 passing through the first splitting surface 160. The first splitting surface 160 may include a non-polarization beam splitter that is not wavelength-dependent instead of a dichroic mirror. When the first image sensor 142 is implemented by the distance image sensor 120 and the third image sensor 146 is implemented by the non-polarization image sensor 70 or 70B, the second splitting surface 166 preferably includes a wavelength-independent non-polarization light beam splitter. By providing a non-polarization beam splitter on the second splitting surface 166, it is possible to suppress a change in the polarization state of the second reflected light 186 reflected by the second splitting surface 166.

The phase difference plate 148A is configured to provide a phase difference to reduce or cancel the phase difference between the s-polarization component and the p-polarization component of the second reflected light 186 produced by at least one of the transmission through the first splitting surface 160, the reflection by the second splitting surface 166, or the reflection by the second incidence surface 164. The phase difference plate 148A compensates for a change in the polarization state of the second reflected light 186 caused by the light splitting element 140. In particular, the polarization state of the second reflected light 186 can change considerably when the second reflected light 186 is totally reflected internally by the second incidence surface 164, which is the interface between the prism and the air. The magnitude of the phase difference provided by the phase difference plate 148A does not particularly matter. For example, the phase difference is about 120 degrees.

In the second exemplary configuration of FIG. 17, too, it is possible, by using the light splitting element 140, to image the incident light 180 to obtain a polarization image, a non-polarization image, and a distance image by using the polarization image sensor 20, the non-polarization image sensor 70 or 70B, and the distance image sensor 120, respectively. According to this embodiment, it is possible to suppress a change in the polarization state of the second reflected light 182 produced in at least one of the first splitting surface 160, the second splitting surface 266, or the second incidence surface 164, by providing the phase difference plate 148A between the light splitting element 140 and the polarization image sensor 20 (second image sensor 144). According to this embodiment, a polarization image in which the change in the polarization state in the dichroic mirror is suppressed can be obtained by providing a non-polarization dichroic mirror on the first splitting surface 160 or the second splitting surface 166. According to this embodiment, a polarization image in which the change in the polarization state in the beam splitter is suppressed can be obtained by providing a non-polarization light beam splitter on the first splitting surface 160 or the second splitting surface 166.

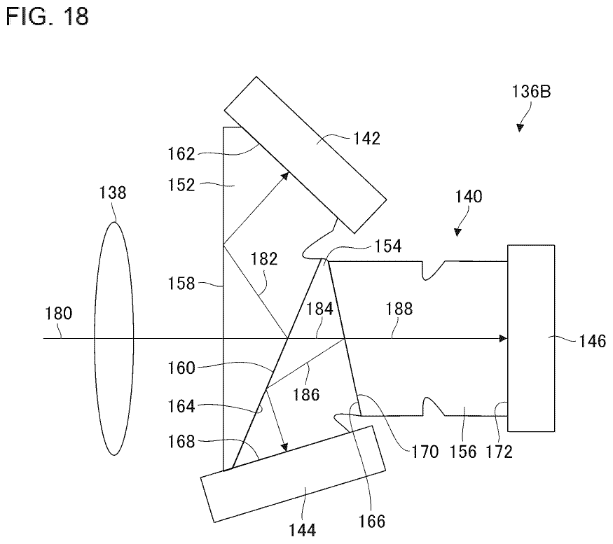

FIG. 18 schematically shows an imaging unit 136A according to the third exemplary configuration of the fifth embodiment. The third exemplary configuration differs from the first exemplary configuration and the second exemplary configuration described above in that the phase difference plate 148, 148A is not provided. The following description of the third exemplary configuration highlights the difference from the first exemplary configuration or the second exemplary configuration. A description of common features is omitted as appropriate.

The imaging unit 136B includes a first image sensor 142, a second image sensor 144, a third image sensor 146, and a light splitting element 140. In the third exemplary configuration, the third image sensor 146 is implemented by the polarization image sensor 20. In the third exemplary configuration, one of the first image sensor 142 and the second image sensor 144 is implemented by the non-polarization image sensor 70 or 70B, and the other of the first image sensor 142 and the second image sensor 144 is implemented by the distance image sensor 120.

When the first image sensor 142 is implemented by the non-polarization image sensor 70 or 70B and the second image sensor 144 is implemented by the distance image sensor 120, the first splitting surface 160 preferably includes a wavelength-independent non-polarization beam splitter. By providing a non-polarization light beam splitter on the first splitting surface 160, it is possible to suppress a change in the polarization state of the first transmitted light 184 passing through the first splitting surface 160. When the first image sensor 142 is implemented by the non-polarization image sensor 70 or 70B and the second image sensor 144 is implemented by the distance image sensor 120, the second splitting surface 166 is preferably a non-polarization dichroic mirror. The dichroic mirror provided on the second splitting surface 166 is designed to selectively transmit visible light and selectively reflect infrared light. By providing a non-polarization dichroic mirror on the second splitting surface 166, it is possible to suppress a change in the polarization state of the second transmitted light 188 passing through the second splitting surface 166. The second splitting surface 166 may include a non-polarization beam splitter that is not wavelength-dependent such as a half mirror instead of a dichroic mirror.

When the first image sensor 142 is implemented by the distance image sensor 120 and the second image sensor 144 is implemented by the non-polarization image sensor 70 or 70B, the first splitting surface 160 is preferably a non-polarization dichroic mirror. The dichroic mirror provided on the first splitting surface 160 is designed to selectively transmit visible light and selectively reflect infrared light. By providing a non-polarization dichroic mirror on the first splitting surface 160, it is possible to suppress a change in the polarization state of the first transmitted light 184 passing through the first splitting surface 160. The first splitting surface 160 may include a non-polarization beam splitter that is not wavelength-dependent instead of a dichroic mirror. When the first image sensor 142 is implemented by the distance image sensor 120 and the third image sensor 146 is implemented by the non-polarization image sensor 70 or 70B, the second splitting surface 166 preferably includes a wavelength-independent non-polarization light beam splitter. By providing a non-polarization beam splitter on the second splitting surface 166, it is possible to suppress a change in the polarization state of the second reflected light 186 reflected by the second splitting surface 166.

In the third exemplary configuration, too, it is possible, by using the light splitting element 140, to image the incident light 180 to obtain a polarization image, a non-polarization image, and a distance image by using the polarization image sensor 20, the non-polarization image sensor 70 or 70B, and the distance image sensor 120, respectively. According to this embodiment, it is possible to achieve a configuration in which internal total reflection does not occur on the optical path toward the polarization image sensor 20 by implementing the third image sensor 146 by the polarization image sensor 20. Thereby, a polarized image that is not affected by the change in the polarization state caused by internal total reflection can be obtained. According to this embodiment, it is possible to suppress a change in the polarization state of the second transmitted light 188 caused by the transmission through the first splitting surface 160 and the second splitting surface 166 by providing a non-polarization dichroic mirror or beam splitter on the first splitting surface 160 and the second splitting surface 166. Thereby, an appropriate polarization image can be obtained without providing a phase difference plate between the light splitting element 140 and the polarization image sensor 20 (third image sensor 146).

### Sixth Embodiment

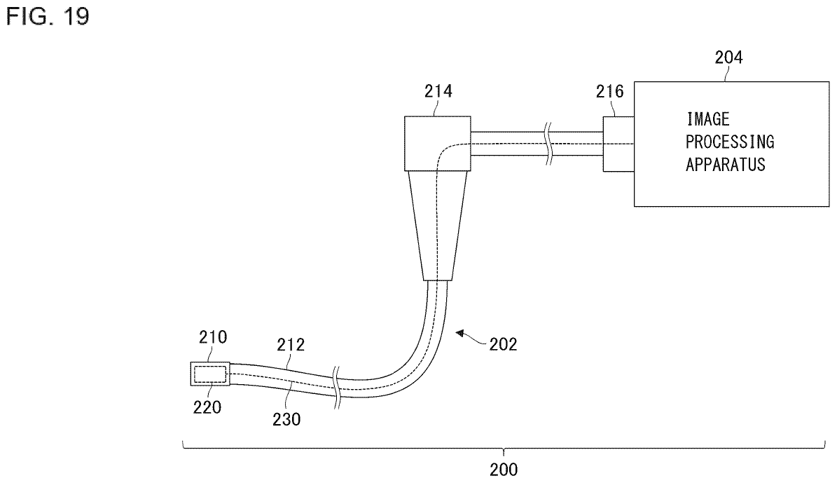

FIG. 19 schematically shows a configuration of an endoscopic system 200 according to the sixth embodiment. The endoscopic system 200 includes an endoscope 202 and an image processing apparatus 204. The endoscope 202 includes an inserted portion 212 having a tip portion 210, a manipulation portion 214, and a connection portion 216.

The inserted portion 212 is a portion inserted inside a subject of observation. The inserted portion 212 is made of, for example, a member including flexibility and is configured such that the orientation of the tip portion 210 can be adjusted by bending the neighborhood of the tip portion 210. In this case, the endoscope 202 is configured as a flexible scope. The inserted portion 212 may be made of a member that does not have flexibility. In this case, the endoscope 202 is configured as a rigid scope.

The tip portion 210 is a portion directed toward a subject inside the target of observation and is provided at the tip of the inserted portion 212. An imaging unit 220 is provided inside the tip portion 210.

The manipulation portion 214 is a portion grasped by a user using the endoscope 202. The manipulation portion 214 is provided with a manipulation knob (not shown) for changing the orientation of the tip portion 210.

The connection portion 216 is an interface for connecting the endoscope 202 to the image processing apparatus 204. The image signal output from the imaging unit 220 is transmitted to the image processing apparatus 204 via the connection portion 216 through a transmission cable 230 provided inside the inserted portion 212 and the manipulation portion 214.

The image processing apparatus 204 executes an image process that uses an image signal output from the imaging unit 220 and transmitted through the transmission cable 230 as an input.

The endoscopic system 200 can include any of the imaging apparatuses 10, 10A, 10B, 10C, and 10D according to the above-described embodiments. The imaging unit 220 can be configured in the same manner as the imaging units 12, 12A, 12B, 12C, 12D, 136, 136A, 136B according to the above-described embodiments. The image processing apparatus 204 can be configured in the same manner as any of the image processing apparatuses 14, 14A, 14B, 14C, and 14D according to the above-described embodiments.

The transmission cable 230 can transmit at least one of the first image signals 36, the second image signal 86, 86B, and the distance image signal 118 output from the imaging unit 220 to the image processing apparatus 204. The transmission cable 230 can transmit the pulse signal 116 from the image processing apparatus 204 to the imaging unit 220.

According to this embodiment, a clearer color image can be generated by using the image signal acquired from the imaging unit 220 provided in the tip portion 210 of the endoscope 202. For example, a color image with suppressed overexposure and underexposure can be generated by synthesizing the RGB values of the four polarization components. Further, a high-resolution color image can be generated by adding the high-frequency component obtained from the output signal of the non-polarization image sensor 70 and 70B to the color image obtained from the output signal of the polarization image sensor 20. Thereby, a color image with a high resolution and suppressed aliasing distortion can be generated. Further, a more suitable polarized color image with a high resolution and suppressed aliasing distortion can be generated by adding the high-frequency component to the color image of each of the four polarization components. Further, point cloud data that associates the coordinate values indicating the three-dimensional position of the subject with the RGB value based on a clear color image can be generated by further using the distance image sensor. Thereby, a more suitable three-dimensional model of the subject can be generated.

### Seventh Embodiment

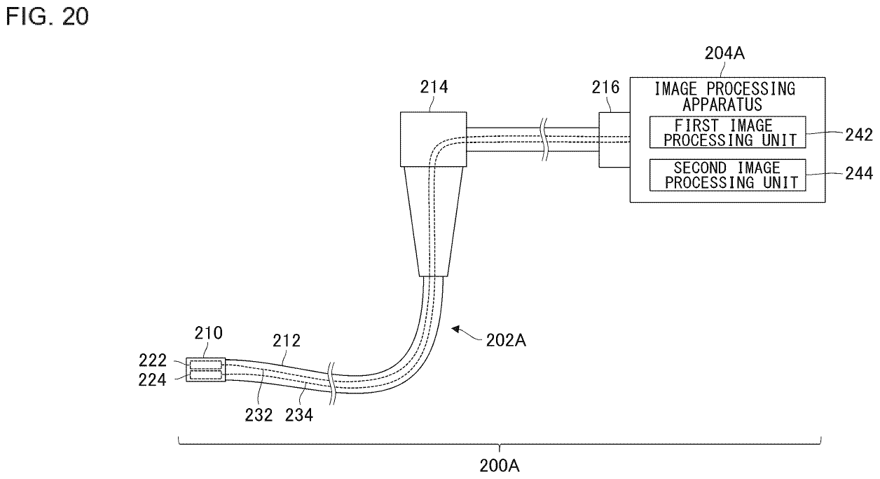

FIG. 20 schematically shows a configuration of an endoscopic system 200A according to the seventh embodiment. The endoscopic system 200A according to the seventh embodiment differs from the sixth embodiment described above in that the endoscopic system 200A is configured to be of a binocular type. The following description of the seventh embodiment highlights the difference from the sixth embodiment. A description of common features is omitted as appropriate. The features identical to those of the sixth embodiment are denoted by identical reference symbols in the drawings.

The endoscopic system 200A includes an endoscope 202A and an image processing apparatus 204A. The endoscope 202A includes an inserted portion 212 including a tip portion 210, a manipulation portion 214, and a connection portion 216.

A first imaging unit 222 and a second imaging unit 224 are provided inside the tip portion 210. The first imaging unit 222 and the second imaging unit 224 are, for example, provided in parallel on the left and the right. Each of the first imaging unit 222 and the second imaging unit 224 can be configured in the same manner as the imaging unit 12, 12A, 12B, 12C, 12D, 136, 136A, 136B according to the above-described embodiments.

A first transmission cable 232 and a second transmission cable 234 are provided inside the inserted portion 212 and the manipulation portion 214. The first transmission cable 232 transmits the image signal, etc. output from the first imaging unit 222. The second transmission cable 234 transmits the image signal, etc. output from the second imaging unit 224.

The image processing apparatus 204A includes a first image processing unit 242 and a second image processing unit 244. The first image processing unit 242 executes an image process that uses the image signal output from the first imaging unit 222 and transmitted through the first transmission cable 232 as an input. The second image processing unit 244 executes an image process that uses the image signal output from the second imaging unit 224 and transmitted through the second transmission cable 234 as an input. Each of the first image processing unit 242 and the second image processing unit 244 can be configured in the same manner as any of the image processing apparatuses 14, 14A, 14B, 14C, 14D according to the above-described embodiments.

According to this embodiment, a stereo image can be generated by using the image signal acquired from the first imaging unit 222 and the second imaging unit 224 provided in the tip portion 210 of the endoscope 202A. According to this embodiment, each of the left and right color images constituting the stereo image can be a clearer color image.

According to the present disclosure, a clear image can be generated by using a polarization image sensor.

The present disclosure has been explained with reference to the embodiments described above, but the present disclosure is not limited to the embodiments described above, and appropriate combinations or replacements of the features shown in the examples presented are also encompassed by the present disclosure.

Some embodiments of the present disclosure will now be described.

An imaging apparatus according to a first embodiment of the present disclosure includes: a polarization image sensor in which pixel groups, each including 2×2 pixels for detecting four polarization components that vary depending on a pixel, are in a two-dimensional arrangement, and in which RGB color filters are in a Bayer arrangement, each RGB filter being arranged in each pixel group, an interpolation processing unit that decomposes a pixel value output from the polarization image sensor into each polarization component, generates four Bayer images corresponding to the four polarization components, and calculates, pixel by pixel, an RGB value of each of the four polarization components by debayering and upconverting each of the four Bayer images; a brightness calculation unit that calculates, pixel by pixel, a brightness value of each of the four polarization components from the RGB value of each of the four polarization components; a rank processing unit that ranks, pixel by pixel, the four polarization components in an order of magnitude of the brightness values of the four polarization components; a reference generation unit that calculates, pixel by pixel, a reference brightness value obtained by synthesizing brightness values of a plurality of polarization components that at least include a second-place polarization component and a third-place polarization component; and a synthesis processing unit that a) outputs a reference RGB value obtained by synthesizing the RGB values of the plurality of polarization components when the reference brightness value matches a predetermined threshold value, b) outputs a composite RGB value obtained by synthesizing a high-rank RGB value, derived from synthesizing the RGB values of a first-place polarization component and the second-place polarization component, and the reference RGB value when the reference brightness value is smaller than the threshold value, and c) outputs a composite RGB value obtained by synthesizing a low-rank RGB value, derived from synthesizing the RGB values of the third-place polarization component and a fourth-place polarization component, and the reference RGB value when the reference brightness value is greater than the threshold value.

In the first embodiment, the synthesis processing unit b) in a case of a pixel where the reference brightness value is smaller than the threshold value, may increase a weight of the high-rank RGB value in synthesis as the reference brightness value decreases, and b) in a case of a pixel where the reference brightness value is greater than the threshold value, may increase a weight of the low-rank RGB value in synthesis as the reference brightness value increases.

In the first embodiment, the imaging apparatus may further include: a non-polarization image sensor in which pixels are in a two-dimensional arrangement, the number of the pixels of the non-polarization image sensor being greater than the number of arrangements of the pixel groups of the polarization image sensor; a light splitting element that splits an incident light into a light traveling toward the polarization image sensor and a light traveling toward the non-polarization image sensor; a high-frequency extraction unit that extracts a high-frequency component of each pixel by using an output image of the non-polarization image sensor; and an addition processing unit that adds, pixel by pixel, the high-frequency component to the RGB value output from the synthesis processing unit to generate a high-resolution image.

In the first embodiment, the imaging apparatus may further include: a distance image sensor that measures a distance to a subject, a light splitting element that splits an incident light into a light traveling toward the polarization image sensor and a light traveling toward the distance image sensor; a three-dimensional position calculation unit that calculates, pixel by pixel, coordinate values indicating a three-dimensional position of the subject by using an output signal of the distance image sensor; and a point cloud data generation unit that generates point cloud data that associates, pixel by pixel, the RGB value output from the synthesis processing unit with the coordinate values calculated by the three-dimensional position calculation unit.

In the first embodiment, the imaging apparatus may further include: a non-polarization image sensor in which pixels are in a two-dimensional arrangement, the number of the pixels of the non-polarization image sensor being greater than the number of arrangements of the pixel groups of the polarization image sensor; a distance image sensor that measures a distance to a subject, a light splitting element that splits an incident light into a light traveling toward the polarization image sensor, a light traveling toward the non-polarization image sensor, and a light traveling toward the distance image sensor; a high-frequency extraction unit that extracts a high-frequency component of each pixel by using an output image of the non-polarization image sensor; and an addition processing unit that adds, pixel by pixel, the high-frequency component to the RGB value output from the synthesis processing unit to generate a high-resolution image; a three-dimensional position calculation unit that calculates, pixel by pixel, coordinate values indicating a three-dimensional position of the subject by using an output signal of the distance image sensor; and a point cloud data generation unit that generates point cloud data that associates, pixel by pixel, the RGB value of the high-resolution image output from the addition processing unit with the coordinate values calculated by the three-dimensional position calculation unit.

A second embodiment of the present disclosure relates to an imaging apparatus including: a polarization image sensor in which pixel groups, each including 2×2 pixels for detecting four polarization components that vary depending on a pixel, are in a two-dimensional arrangement, and in which RGB color filters are in a Bayer arrangement, each RGB filter being arranged in each pixel group, a non-polarization image sensor in which pixels are in a two-dimensional arrangement, the number of the pixels of the non-polarization image sensor being greater than the number of arrangements of the pixel groups of the polarization image sensor; a light splitting element that splits an incident light into a light traveling toward the polarization image sensor and a light traveling toward the non-polarization image sensor; an interpolation processing unit that decomposes a pixel value output from the polarization image sensor into each polarization component, generates four Bayer images corresponding to the four polarization components, and calculates, pixel by pixel, an RGB value of each of the four polarization components by debayering and upconverting each of the four Bayer images; a high-frequency extraction unit that extracts a high-frequency component of each pixel by using an output image of the non-polarization image sensor; and an addition processing unit that adds, pixel by pixel, the high-frequency component to the RGB value of each of the four polarization components output from the interpolation processing unit and generates four high-resolution images corresponding to the four polarization components.

In the first embodiment or the second embodiment, the non-polarization image sensor may be a color image sensor in which RGB color filters are in a Bayer arrangement, each RGB filter being arranged in each pixel, the high-frequency extraction unit may extract the high-frequency component from the RGB value of each pixel calculated by debayering the Bayer images output from the non-polarization image sensor, and the addition processing unit may add, pixel by pixel, the RGB value indicating the high-frequency component to the RGB value of each of the four polarization components.

In the first embodiment or the second embodiment, the non-polarization image sensor may be a monochrome image sensor not including a color filter, the high-frequency extraction unit may extract the high-frequency component from the brightness value of each pixel output from the monochrome image sensor, and the addition processing unit may add, pixel by pixel, the brightness value indicating the high-frequency component to the RGB value of each of the four polarization components.

In the second embodiment, the imaging apparatus may further include: a distance image sensor that measures a distance to a subject, the light splitting element may split the incident light further into a light traveling toward the distance image sensor, the imaging apparatus further including: a three-dimensional position calculation unit that calculates, pixel by pixel, coordinate values indicating a three-dimensional position of the subject by using an output signal of the distance image sensor; and a point cloud data generation unit that generates point cloud data that associates, pixel by pixel, the RGB value of each of four high-resolution images output from the addition processing unit with the coordinate values calculated by the three-dimensional position calculation unit.

The third embodiment of the present disclosure relates to an endoscopic system including the imaging apparatus of the first embodiment or the second embodiment. The endoscopic system includes an endoscope and an image processing apparatus, wherein the endoscope includes an inserted portion having a tip portion directed toward a subject, a transmission cable is provided inside the inserted portion, and the image processing apparatus acquires a signal via the transmission cable.

In the third embodiment, the polarization image sensor may be provided inside the tip portion, the transmission cable may transmit the output signal of the polarization image sensor, and the image processing apparatus may include the interpolation processing unit, the brightness calculation unit, the rank processing unit, the reference generation unit, and the synthesis processing unit and acquire the output signal of the polarization image sensor.

In the third embodiment, the polarization image sensor, the non-polarization image sensor, and the light-splitting element may be provided inside the tip portion, the transmission cable may transmit an output signal of the polarization image sensor and the non-polarization image sensor, and the image processing apparatus may include the interpolation processing unit, the high-frequency extraction unit, and the addition processing unit and acquire the output signal of the polarization image sensor and the non-polarization image sensor.

What is claimed is:

1. An imaging apparatus comprising:
a polarization image sensor in which pixel groups, each including 2×2 pixels for detecting four polarization components that vary depending on a pixel, are in a two-dimensional arrangement, and in which RGB color filters are in a Bayer arrangement, each RGB filter being arranged in each pixel group,
an interpolation processing unit that decomposes a pixel value output from the polarization image sensor into each polarization component, generates four Bayer images corresponding to the four polarization components, and calculates, pixel by pixel, an RGB value of each of the four polarization components by debayering and upconverting each of the four Bayer images;
a brightness calculation unit that calculates, pixel by pixel, a brightness value of each of the four polarization components from the RGB value of each of the four polarization components;
a rank processing unit that ranks, pixel by pixel, the four polarization components in an order of magnitude of the brightness values of the four polarization components;
a reference generation unit that calculates, pixel by pixel, a reference brightness value obtained by synthesizing brightness values of a plurality of polarization components that at least include a second-place polarization component and a third-place polarization component; and
a synthesis processing unit that a) outputs a reference RGB value obtained by synthesizing the RGB values of the plurality of polarization components when the reference brightness value matches a predetermined threshold value, b) outputs a composite RGB value obtained by synthesizing a high-rank RGB value, derived from synthesizing the RGB values of a first-place polarization component and the second-place polarization component, and the reference RGB value when the reference brightness value is smaller than the threshold value, and c) outputs a composite RGB value obtained by synthesizing a low-rank RGB value, derived from synthesizing the RGB values of the third-place polarization component and a fourth-place polarization component, and the reference RGB value when the reference brightness value is greater than the threshold value.

2. The imaging apparatus according to claim 1,
wherein the synthesis processing unit b) in a case of a pixel where the reference brightness value is smaller than the threshold value, increases a weight of the high-rank RGB value in synthesis as the reference brightness value decreases, and c) in a case of a pixel where the reference brightness value is greater than the threshold value, increases a weight of the low-rank RGB value in synthesis as the reference brightness value increases.

3. The imaging apparatus according to claim 1, further comprising:
a non-polarization image sensor in which pixels are in a two-dimensional arrangement, the number of the pixels of the non-polarization image sensor being greater than the number of arrangements of the pixel groups of the polarization image sensor;
a light splitting element that splits an incident light into a light traveling toward the polarization image sensor and a light traveling toward the non-polarization image sensor;
a high-frequency extraction unit that extracts a high-frequency component of each pixel by using an output image of the non-polarization image sensor; and
an addition processing unit that adds, pixel by pixel, the high-frequency component to the RGB value output from the synthesis processing unit to generate a high-resolution image.

4. The imaging apparatus according to claim 1, further comprising:
a light splitting element that splits an incident light into a light traveling toward the polarization image sensor and a light traveling toward the non-polarization image sensor;
a high-frequency extraction unit that extracts a high-frequency component of each pixel by using an output image of the non-polarization image sensor; and
an addition processing unit that adds, pixel by pixel, the high-frequency component to the RGB value of each of the four polarization components output from the interpolation processing unit and generates four high-resolution images corresponding to the four polarization components.

5. The imaging apparatus according to claim 3,
wherein the non-polarization image sensor is a color image sensor in which RGB color filters are in a Bayer arrangement, each RGB filter being arranged in each pixel,
wherein the high-frequency extraction unit extracts the high-frequency component from the RGB value of each pixel calculated by debayering the Bayer images output from the non-polarization image sensor, and
wherein the addition processing unit adds, pixel by pixel, the RGB value indicating the high-frequency component to the RGB value of each of the four polarization components.

6. The imaging apparatus according to claim 3,
wherein the non-polarization image sensor is a monochrome image sensor not including a color filter,
wherein the high-frequency extraction unit extracts the high-frequency component from the brightness value of each pixel output from the monochrome image sensor, and
wherein the addition processing unit adds, pixel by pixel, the brightness value indicating the high-frequency component to the RGB value of each of the four polarization components.

7. The imaging apparatus according to claim 3, further comprising:
a distance image sensor that measures a distance to a subject,
wherein the light splitting element splits the incident light further into a light traveling toward the distance image sensor,
the imaging apparatus further comprising:
a three-dimensional position calculation unit that calculates, pixel by pixel, coordinate values indicating a three-dimensional position of the subject by using an output signal of the distance image sensor; and
a point cloud data generation unit that generates point cloud data that associates, pixel by pixel, the RGB value of each of four high-resolution images output from the addition processing unit with the coordinate values calculated by the three-dimensional position calculation unit.

8. The imaging apparatus according to claim 1, further comprising:
a distance image sensor that measures a distance to a subject;
a light splitting element that splits an incident light into a light traveling toward the polarization image sensor and a light traveling toward the distance image sensor;
a three-dimensional position calculation unit that calculates, pixel by pixel, coordinate values indicating a three-dimensional position of the subject by using an output signal of the distance image sensor; and
a point cloud data generation unit that generates point cloud data that associates, pixel by pixel, the RGB value output from the synthesis processing unit with the coordinate values calculated by the three-dimensional position calculation unit.

9. An endoscopic system including the imaging apparatus according to claim 1, comprising:
an endoscope including an inserted portion having a tip portion directed toward a subject, the polarization image sensor being provided inside the tip portion, and a transmission cable for transmitting an output signal of the polarization image sensor being provided inside the inserted portion; and
an image processing apparatus including the interpolation processing unit, the brightness calculation unit, the rank processing unit, the reference generation unit, and the synthesis processing unit, the image processing apparatus being configured to acquire the output signal via the transmission cable.

10. An endoscopic system including the imaging apparatus according to claim 3, comprising:
an endoscope including an inserted portion having a tip portion directed toward a subject, the polarization image sensor, the non-polarization image sensor, and the light splitting element being provided inside the tip portion, and a transmission cable for transmitting an output signal of the polarization image sensor and the non-polarization image sensor being provided inside the inserted portion; and
an image processing apparatus including the interpolation processing unit, the high-frequency extraction unit, and the addition processing unit, the image processing apparatus being configured to acquire the output signal via the transmission cable.
